(19) United States
(12) Patent Application Publication
(10) Pub. No.: US 2024/0344955 A1
(43) Pub. Date: Oct. 17, 2024

(54) REACTIVE CONDENSATION PARTICLE COUNTER FOR THE DETECTION OF TRACE ATMOSPHERIC GASES

(71) Applicant: Carnegie Mellon University, Pittsburgh, PA (US)

(72) Inventors: Coty JEN, Pittsburgh, PA (US); Dominic CASALNUOVO, Pittsburgh, PA (US); Darren CHENG, Pittsburgh, PA (US)

(21) Appl. No.: 18/441,844

(22) Filed: Feb. 14, 2024

Related U.S. Application Data
(60) Provisional application No. 63/445,333, filed on Feb. 14, 2023.

(51) Int. Cl. G01N 15/06; G01N 15/00; G01N 33/00

(52) U.S. Cl. CPC G01N 15/065; G01N 2015/0038; G01N 2015/0681; G01N 33/005

(57) ABSTRACT

Systems and devices to measure atmospheric gaseous reactive gases that may form atmospheric aerosol particles may generally includes a reactor in fluid communication with a particle counter. The reactor may include a first inlet configured to receive a continuous flow of a first fluid and a second inlet configured to receive a continuous flow of a second fluid. The reactor may include a first outlet in fluid communication with the particle counter to detect a reaction product of the first fluid and second fluid. The reaction product may include 1-3 nanometer particles. The reactor may include a second outlet to exhaust any remaining portion of the reaction product flow from the reactor. Methods of making and using the systems and devices to measure atmospheric gaseous reactive gases that may form atmospheric aerosol particles are also described.

### CROSS-REFERENCE TO RELATED APPLICATION

This application claims the benefit of U.S. Provisional Application No. 63/445,333, filed on Feb. 14, 2023, the entire contents of which is incorporated herein by reference.

### GOVERNMENT RIGHTS

This invention was made with U.S. government support under DE-SC0022224 awarded by the Department of Energy (DOE). The government has certain rights in the invention.

### BACKGROUND

Atmospheric aerosol particles are emitted directly as primary particles or form via nucleation of gas-phase molecules into stable secondary particles. Both primary and secondary particles play an important role in the Earth's radiative balance and climate as these particles may serve as cloud condensation nuclei. Atmospheric nucleation is the process whereby gaseous precursor compounds react to form stable particles about 0.1-10 nm in diameter, such as 1-nm in diameter. These particles may grow to larger sizes via condensation of other compounds. About 50% of global seed particles for cloud droplet formation originate from atmospheric nucleation processes. Nucleation has been observed in polluted and pristine conditions at ground level and in the upper boundary layer of the atmosphere. Sulfuric acid may be a major driver of global atmospheric nucleation reactions with other compounds, such as ammonia and amines, for example, by helping form stable particles. The rate at which sulfuric acid forms atmospheric particles may depend on the sulfuric acid concentration to the 2-3 power. Accurately predicting atmospheric nucleation and subsequent particle size distributions may be important to understanding how aerosol particles influence cloud properties and, thus, Earth's radiative balance and climate.

Accordingly, more efficient and/or cost-effective devices, systems, and methods to detect and measure atmospheric aerosol particles and precursor gases may be desirable.

### SUMMARY

The present invention is directed to more efficient and/or cost-effective devices, systems, and methods to detect and measure atmospheric aerosol particles and precursor gases that form aerosol particles.

Systems and devices to measure atmospheric gaseous reactive gases that may form atmospheric aerosol particles may generally comprise a reactor in fluid communication with a particle counter. The reactor may comprise a first inlet configured to receive a continuous flow of a first fluid and a second inlet configured to receive a continuous flow of a second fluid. The reactor may comprise a first outlet in fluid communication with the particle counter to detect a reaction product of the first fluid and second fluid. The reaction product may comprise 1-3 nanometer particles. The reactor may comprise a second outlet to exhaust any remaining portion of the reaction product flow from the reactor.

Methods of measuring atmospheric reactive precursor gases that form aerosol particles may generally comprise using systems and devices comprising a reactor in fluid communication with a particle counter, wherein the reactor may comprise a first inlet configured to receive a continuous flow of a first fluid and a second inlet configured to receive a continuous flow of a second fluid; wherein the reactor may comprise a first outlet in fluid communication with the particle counter to detect a reaction product of the first fluid and second fluid; wherein the reaction product may comprise 1-3 nanometer particles; and/or wherein the reactor may comprise a second outlet to exhaust any remaining portion of the reaction product flow from the reactor.

Methods of measuring atmospheric reactive precursor gases that form aerosol particles may generally comprise contacting a continuous flow of a first fluid and a continuous flow of a second fluid to generate a nanometer particle comprising a reaction product of the first fluid and second fluid; and detecting the particle via a condensation particle counter. The first fluid may comprise one of an amine and an atmospheric gas comprising at least one reactant, and the second fluid may comprise the other of the amine and the atmospheric gas comprising at least one reactant. The reactant may comprise at least one of sulfuric acid, dimethylamine, trimethylamine, and diamine), and the amine may comprise at least one of dimethylamine, ethylenediamine, trimethylamine, and 1,4-butanediamine. The reaction product may comprise at least one of sulfuric acid-dimethylamine particles, sulfuric acid-ethylene diamine particles, sulfuric acid-trimethylamine particles, and sulfuric acid-1,4-butanediamine particles.

### BRIEF DESCRIPTION OF THE FIGURES

The devices, systems, and processes described herein may be better understood by considering the following description in conjunction with the accompanying drawings; it being understood that this disclosure is not limited to the accompanying drawings.

FIG. 1 includes a diagram of the sulfuric acid dimethylamine-reactive condensation particle counter (SAD-RCPC) according to the present invention. The SAD-RCPC may comprise a length of 44 cm and a diameter of 5 cm. For the High Dilution (HD) configuration, the main flow may comprise an inert gas entrained with dimethylamine and water vapor. The main flow may comprise a flowrate of 0.1-10 Liters per minute (LPM), such as 0.8-1.2 LPM. For example, the main flowrate may be 1.0±0.5 LPM, 2.0±0.5 LPM, 5.0±0.5 LPM, or 9.5±0.5 LPM. The side flow may comprise the sample gas including sulfuric acid. The side flow comprises a flowrate of 0.01-1.0 LPM, such as, for example, 0.035-0.1 LPM, 0.01 LPM, 0.035 LPM, 0.05 LPM, 0.1, LPM, 0.5 LPM, or 0.8 LPM. In the Low Dilution (LD) configuration, the main flow may comprise the sample gas including sulfuric acid having a flowrate of 0.9 LPM and the side flow may comprise dimethylamine vapor in an inert, particle-free carrier gas having a flowrate of 0.1 LPM. The process flow to the condensation particle counter may be 0.1-1.0 LPM, such as 0.3 LPM, for example.

FIGS. 2A and 2B include a chart showing CPC measured particle concentrations compared to measured sulfuric acid (SA) concentrations compared for (A) high dilution (HD) and (B) low dilution (LD) configurations. Particles are measured using a MAGIC water condensation particle counter with a two-stage diethylene glycol inlet (DEG wCPC), and sulfuric acid is measured with a nitrate or acetate chemical ionization mass spectrometer (CIMS). Measurements with nitrogen as a carrier gas are shown as squares and particle-free air as circles.

FIGS. 3A and 3B includes a comparison chart showing SAD-RCPC sulfuric acid (SA) and CIMS SA concentrations calculated using the (A) reaction model and (B) statistical model. Measurements with nitrogen as a carrier gas are shown as squares and particle free air are shown as circles. High Dilution (HD) configuration measurements are shown as solid points and Low Dilution (LD) configuration measurements are shown as hollow points.

FIG. 4 includes a comparison chart showing SAD-RCPC sulfuric acid (SA) and CIMS SA concentrations using the reaction model. Measurements with squares are nitrogen as a carrier gas and circles are particle-free compressed air. The gray color scale provides relative humidity (RH).

FIG. 5 includes a chart showing predicted cluster concentrations produced from reactions of sulfuric acid and dimethylamine following dilution amounts for the LD configuration.

FIG. 6 includes a chart showing total particle concentration estimated by the SAD-RCPC reaction model assuming three different detection efficiencies. Case 1: (N2) 0.1% [N2]+30% [N3]+100% [N≥4]; Case 2: [N≥4], (N3) 30% [N3]+100% [N≥4]; and Case 3: (N4) 100% [N≥4].

FIG. 7 includes a chart showing detection efficiency curve of the DEG-wCPC for size-selected, electrically positive, ammonium sulfate particles measured by a differential mobility analyzer.

FIG. 8 includes a diagram of the sulfuric acid dimethylamine-reactive condensation particle counter (SAD-RCPC) of FIG. 1 showing flowrate for the main flow, side flow, and process flow.

FIG. 9 includes a diagram of the shortened aluminum reactor for the SAD-RCPC having a 1.5 inch long cone-shaped mixing chamber comprising a first inlet to receive sample gas at a flowrate of 0.3-0.6 LPM and a second inlet to receive a reagent (such as DMA) at a flowrate of 0.035-0.1 LPM, and a 5 inch long reaction chamber (1.31 inch inner diameter and 1.5 inch outer diameter) having a first outlet to release process flow at a flowrate of 0.3 LPM and a second outlet to release exhaust.

FIG. 10 includes an isometric view of a SAD-RCPC comprising a core flow reactor coupled to the DEG-wCPC (c.f., FIG. 1) The core flow reactor introduces sampled air including sulfuric acid through the center, and particle-free clean air including dimethylamine vapor is introduced along the walls of the reactor and parallel to the sampled air stream.

FIG. 11 includes a side view of the core flow reactor and flowrates of the sample gas (1-2 LPM), sheath gas (1-8 times the sample gas flowrate, 8-16 LPM), and pre-existing particle exhaust flow (1-4 times the sample gas flowrate, 1-8 LPM) flow rates. The dimethylamine vapor flow is flow straightened using metal screens as shown as vertical lines on the left side of FIG. 11.

FIG. 12 includes an isometric view of the center flow extractor of the core flow reactor for the SAD-RCPC. The center flow extractor is shown in FIG. 10 and FIG. 11. The extractor may remove the sample air stream without disturbing the particle-free dimethylamine outer flow.

FIG. 13 includes a schematic of the SAD-RCPC comprising a nucleation flow reactor shown in FIG. 1 coupled to a DEG wCPC and a pulse height analysis (shown in the right graph) of Pittsburgh, PA air demonstrating the separation and detection of particles formed in the flow reactor from ambient particles.

FIG. 14 includes a schematic of an acid-base reaction model of SA monomer (SA) nucleating with dimethylamine (DiMA) to form a dimer (N2), a trimer (N3), and a tetramer (N4) which is a 1 nm particle. The inputs to the model are the nucleation time, 1-nm particle concentration, and dimethylamine concentration (boxed DiMA). The model outputs sampled SA concentration (boxed SA).

FIG. 15A and FIG. 15B include a deployable TBS-mounted DEG-wCPCs according to the present invention to measure an effective base concentration ([B_{eff}]) and sulfuric acid (SA) concentration.

FIG. 16 includes a schematic of a base-CPC according to the present invention to measure the effective base concentration, [B_{eff}]. The base-CPC comprises a sulfuric acid flow reactor and a 1-nm versatile water condensation particle counter (vWCPC). Any 1-nm CPC may be used instead of the vWCPC such as the DEG-wCPC. Teflon mesh is used to straighten the flow but is not required if sulfuric acid-base nucleation reaction times are known. FIG. 17 includes a chart showing individual and combinations of stabilizing

compounds sampled by the base-CPC, and the resulting [B_{eff}] as a function of measured base concentration. The mixtures include ammonia, methylamine, dimethylamine, trimethylamine (with this mixture called Base Mix), methanesulfonic acid (MSA), oxal acid, formic acid, acetic acid (with this mixture called OA), and particle-free room air.

FIG. 18 includes a chart showing total observed particle concentration larger than 1-nm produced from reactions of sulfuric, oxalic, malonic, or formic acid and dimethylamine (DMA) or ethylene diamine (EDA). This chart shows that dimethylamine and ethylene diamine may be used for nucleating compound for sulfuric acid in SAD-RCPC. Stabilizing compounds, such as dimethylamine, react with sulfuric acid at the gas-molecule collision limit and does not appreciably react to form particles with other atmospherically relevant compounds.

FIG. 19 includes a pulse height analysis of Pittsburgh, PA air demonstrating the separation and detection of smaller particles formed in the flow reactor according to the present invention from larger ambient particles.

### DETAILED DESCRIPTION

This disclosure generally describes devices and systems to detect and measure the precursor gases that nucleate to form atmospherics aerosol particles as well as methods of making and using the same. It is understood, however, that this disclosure also embraces numerous alternative features, aspects, and advantages that may be accomplished by combining any of the various features, aspects, and/or advantages described herein in any combination or sub-combination that one of ordinary skill in the art may find useful. Such combinations or sub-combinations are intended to be included within the scope of this disclosure. As such, the claims may be amended to recite any features, aspects, and advantages expressly or inherently described in, or otherwise expressly or inherently supported by, this disclosure. Further, any features, aspects, and advantages that may be present in the prior art may be affirmatively disclaimed. Accordingly, this disclosure may comprise, consist of, consist essentially, or be characterized by one or more of the features, aspects, and advantages described herein. As used herein, the term “and/or” includes any and all combinations of one or more of the associated listed items.

All numerical quantities stated herein are approximate, unless stated otherwise. Accordingly, the term “about” may be inferred when not expressly stated. The numerical quantities disclosed herein are to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the terms “about” refers to values within an order of magnitude, potentially within 5-fold or 2-fold of a given value. Notwithstanding the approximations of numerical quantities stated herein, the numerical quantities described in specific examples of actual measured values are reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges are continuous and include every value between the minimum and maximum values. Any maximum numerical limitation recited herein is intended to include all lower numerical limitations. Any minimum numerical limitation recited herein is intended to include all higher numerical limitations.

All compositional ranges stated herein are limited in total to and do not exceed 100 percent (e.g., volume percent or weight percent) in practice. When multiple components may be present in a composition, the sum of the maximum amounts of each component may exceed 100 percent, with the understanding that, and as those skilled in the art would readily understand, that the amounts of the components may be selected to achieve the maximum of 100 percent.

In the following description, certain details are set forth in order to provide a better understanding of various features, aspects, and advantages the invention. However, one skilled in the art will understand that these features, aspects, and advantages may be practiced without these details. In other instances, well-known structures, methods, and/or processes associated with methods of practicing the various features, aspects, and advantages may not be shown or described in detail to avoid unnecessarily obscuring descriptions of other details of the invention.

The terminology used herein is for the purpose of describing particular examples only and is not intended to be limiting. As used herein, the singular forms “a,” “an”, and “the” may be intended to include the plural forms as well, unless the context clearly indicates otherwise. The terms “comprises”, “comprising”, “including”, “having”, and “characterized by”, are inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms are to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected, or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.).

Although the terms first, second, third, etc., may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein may not imply a sequence or order unless clearly indicated by the context. Thus, a first step, clement, component, region, layer, or section discussed below may be termed a second step, element, component, region, layer, or section without departing from the teachings herein.

Spatially or temporally relative terms, such as “before,” “after,” “inner”, “outer”, “beneath”, “below”, “lower”, “above”, “upper”, and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures. As used herein, “top” means furthest away from the substrate, while “bottom” means closest to the substrate. Where a first layer is described as “disposed over,” “provided over,” or “deposited over” a second layer, the first layer is disposed further away from substrate. There may be other layers between the first and second layer, unless it is specified that the first layer is “in contact with,” “disposed on,” “provided on,” or “deposited on” the second layer.

Atmospheric aerosol particles play an important role in the Earth's radiative balance through cloud formation and direct solar radiation scattering. It is believed that sulfuric acid-driven nucleation is a major source of atmospheric aerosol particles and cloud condensation nuclei. However, conventional instrumentation for sulfuric acid is challenging to conduct at high spatial and temporal resolution measurements which are needed to help constrain climate models. The present invention is directed to a Sulfuric Acid Dimethylamine-Reactive Condensation Particle Counter (SAD-RCPC) device. The device may be a portable and cost-effective instrument to measure gaseous compounds that form aerosol particles. These gases include, for example, sulfuric acid vapor. Comparison of the SAD-RCPC with conventional devices, such as the chemical ionization mass spectrometer, demonstrates the SAD-RCPC may accurately measure sulfuric acid concentrations from 10⁶ to 10⁹ molecules cm⁻³ in a particle-free sample gas. The device may be applicable to outdoor measurements where the sample gas also contains background levels of aerosol particles. The device may decrease the barriers in measuring sulfuric acid and/or provide higher resolution measurements relative to conventional devices to reduce uncertainty when evaluating Earth system models.

The formation rate of stable aerosol particles (i.e., nucleation rate) may relate to the sulfuric acid concentration to the power of 2 to 3 across diverse locations. Furthermore, sulfuric acid concentrations vary widely by time of day and proximity to sources. Sulfuric acid concentration concentrations may be from below the detection limit (<10⁴ molecules cm⁻³) to 10⁸ molecules cm⁻³. This large range in sulfuric acid concentrations may cause nucleation rates to fluctuate by many orders magnitude over small spatial and temporal scales. Thus, obtaining and using accurate concentrations of sulfuric acid when predicting atmospheric nucleation rates and subsequent aerosol size distributions is desirable.

Conventional aerosol microphysics and climate models estimate sulfuric acid concentrations from sulfur dioxide, the oxidation precursor to sulfuric acid. Sulfur dioxide is emitted from various natural and anthropogenic sources, many of which are cataloged in emission inventories. Translating these surface emissions into spatially resolved concentrations, including into the free troposphere, results in inaccurate estimates of sulfur dioxide concentrations. For example, sulfur dioxide concentrations have been shown to be a factor of 2-6 higher than those measured during the Atmospheric Tomography (ATom) 4 field campaign. Modeled sulfur dioxide concentrations may be converted to sulfuric acid concentration using empirical proxy relationships that account for hydroxyl radical (OH), nitrogen oxides (NOₓ), ozone (O₃), and nitrous acid (HONO) concentrations, concentration and size of background aerosol particles, and ultraviolet radiation. These conversion relationships are dependent on location and likely change over time. Combined, these factors may result in highly uncertain values for the predicted global sulfuric acid concentrations and nucleation rates in climate models.

High spatially and temporally resolved measurements of atmospheric sulfuric acid may be desirable to constrain predicted sulfuric acid concentration and thus, aerosol number concentration in climate models. Conventional atmospheric sulfuric acid measurements may use a chemical ionization mass spectrometer (CIMS). These instruments may exhibit high sensitivity, often down to 10⁴-10⁵ molecules cm⁻³, and may resolve sulfuric acid from other atmospheric trace compounds. However, CIMS may be expensive ($300K-$500K), heavy (50-100 kg), and/or power-intense (1-2 kW) instrument. In addition, maintaining these instruments and processing the data may be laborious and require a full-time scientist. All these traits persuade numerous research groups and monitoring stations from obtaining and deploying CIMS for laboratory and field measurements. As such, only sparse and short duration measurements of sulfuric acid exist for locations around the world. A more compact and less expensive instrument may be desirable to provide a compressive observational data set for constraining how atmospheric nucleation and aerosols influence Earth's radiative budget.

The present invention is directed to Sulfuric Acid Dimethylamine-Reactive Condensation Particle Counter (SAD-RCPC) device. The device may provide an alternative method to measure atmospheric sulfuric acid concentrations. The device leverages the nucleation reactions of sulfuric acid and dimethylamine to estimate gaseous sulfuric acid concentrations. The SAD-RCPC may comprise a 1-nm condensation particle counter (CPC) and a compact nucleation flow reactor that reacts gaseous sulfuric acid with dimethylamine to form detectable 1-nm particles. Laboratory validation of the SAD-RCPC with a nitrate CIMS demonstrates the feasibility of the SAD-RCPC instrument. Ultimately, the cost, case of use, portability, and robustness of the SAD-RCPC may allow it to be deployed more widely, such as for long-term measurements or on unmanned aerial vehicles, for example. The device may facilitate the measurements to help constrain aerosol microphysics models and provide new information on how nucleation occurs in the boundary layer.

### Sulfuric Acid Dimethylamine-Reactive Condensation Particle Counter (SAD-RCPC)

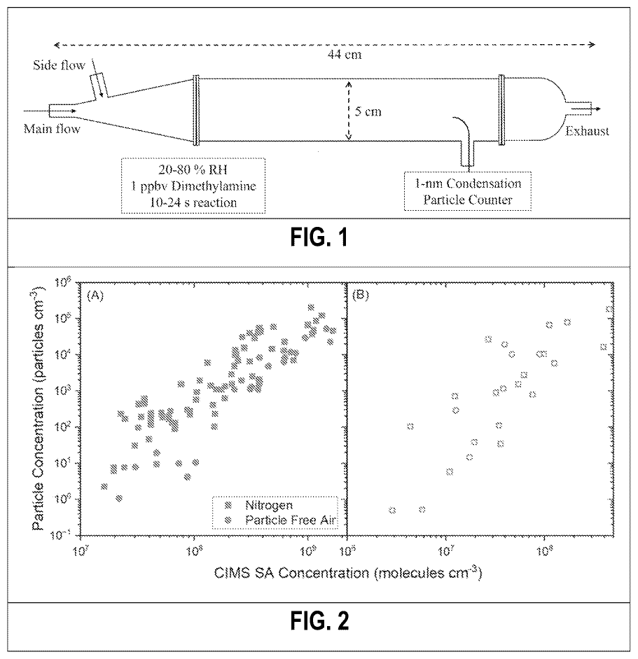

Referring to FIG. 1, the Sulfuric Acid Dimethylamine-Reactive Condensation Particle Counter (SAD-RCPC) comprises a nucleation flow reactor and a 1-nm condensation particle

$$SA + DMA {\to }^{{k}_{f}} {N}_{1} \qquad ( R 1 )$$

$${N}_{1} + {N}_{1} {\to }^{{k}_{f}} {N}_{2} \qquad ( R 2 )$$

$${N}_{2} + {N}_{1} {\to }^{{k}_{f}} {N}_{3} \qquad ( R 3 )$$

$${N}_{3} + {N}_{1} {\to }^{{k}_{f}} {N}_{4} \qquad ( R 4 )$$

$${N}_{2} + {N}_{2} {\to }^{{k}_{f}} {N}_{4} \qquad ( R 5 )$$

$${N}_{i} + {N}_{j} {\to }^{{k}_{f}} {N}_{> 4} for i + j > 4 \qquad ( R 6 )$$

counter (CPC). The Sulfuric Acid Dimethylamine Reactive Condensation Particle Counter (SAD-RCPC) operates by reacting sulfuric acid (SA) (with dimethylamine in excess) with a stabilizing compound in a controlled manner to form detectable 1-nm particles. The stabilizing compound reacts and nucleates with SA at the gas collision limit to maximize the amount of SA converted into particles. In addition, the stabilizing compound may not appreciably nucleate with any other atmospherically relevant compounds on the nucleation timescales utilized in the SAD-RCPC. Dimethylamine (DMA, (CH3)2NH) may not nucleate with water, itself, or other atmospherically relevant acids, such as, for example, formic, malonic, acetic, and oxalic acids, at temperatures greater than 273 K. In addition, SA-DMA nucleation may be the rate limiting step the formation of SA.DMA. With DMA concentration in excess, SA may form 1-nm particles at the SA collision limit. Bases, such as DMA, for example, which react with sulfuric acid and do not nucleate with water, itself, or other atmospherically relevant acids may be used. Examples of bases include other amines, such as ethylene diamine, for example.

Without wishing to be bound to any particular theory, SA-DMA nucleation in the SAD-RCPC may be modeled using an acid-base nucleation reaction scheme shown below (R1)-(R6). This model shows SA-DMA clusters (abbreviated as Ni with i equal to the number of SA or DMA molecules in a cluster) growing to a tetramer, N4 (four SA and 4 DMA molecules) which is up to 1 nm diameter. The sampled (i.e., initial) sulfuric acid concentration may be determined from the differential cluster balance equations generated by the nucleation model when the nucleation time, DMA, and 1-nm particle concentrations are known. The sulfuric acid concentration in the nucleation model may be varied until the modeled particle concentration ([N_{≥4}] is within 5% of the measured particle concentration of the 1-nm CPC. For 1-nm CPCs with low d50 cutoffs, [N3] may be included in the modeled particle concentration with a lower detection efficiency.

Formed clusters that fall outside the model's range may be included in the total particle concentration. The forward rate constant, k, may be the collision rate constant of a SA molecule having an N1 cluster and has a value of 4.2×10⁻¹⁰ cm³ s⁻¹. The same k may be used for all reactions as the changes in collision rate constant with larger clusters in the reactor are small.

The operation and dimension of the nucleation flow reactor may be picked to minimize length while maintaining repeatable flow in order to have a constant nucleation time. Without wishing to be bound to any particular theory, a wide range dimensions and flow rates may be used if the nucleation time is known as this parameter is determined how many particles are formed from sulfuric acid-dimethylamine nucleation reactions. The flow reactor according to the present invention may be 5 cm inner diameter, 44 cm long, and have a 10 cm cone at the entrance of the flow. These dimensions may be changed to adjust the time sulfuric acid-dimethylamine nucleate in the flow reactor. In addition, these dimensions may be changed to match the constraints of the measurement platform. Aerial systems use compact, lightweight instrument; thus, a shorter flow reactor may be used for the SAD-RCPC. Nitrogen gas and 1 ppbv of DMA vapor may be injected in the axial direction at 1 LPM. Water vapor may be also injected with the nitrogen gas to explore effects of relative humidity on the system. High concentrations of DMA (about 1 ppbv) may be generated by flow nitrogen over a DMA permeation tube. Operating the nucleation flow reactor with extremely high excess DMA may reduce/eliminate the need to control the exact concentration of DMA in the reactor. In addition, high concentrations of DMA may reduce/eliminate ammonia or other amines in the sampled gas from appreciably participating in sulfuric acid nucleation. The sampled gas containing sulfuric acid vapor may be injected at an angle in the cone at 0.035 LPM. The angle of injection helps the two flows to mix in the inlet cone. A custom diethylene glycol water condensation particle counter (DEG-wCPC) sampled from the centerline to minimize wall loss using curved stainless-steel tubing. The dilution and reaction time of sulfuric acid in the SAD-RCPC may be estimated using computational fluid dynamics (CFD). The simulations of fluid flow and diffusion in the SAD-RCPC show that sulfuric acid may be diluted by a factor of 25. A reaction time of 24 seconds may be calculated using a transient simulation.

The DEG-wCPC comprises of a custom-built inlet connected to a MAGIC water CPC (Aerosol Dynamics Inc.). This instrument may be used to detect freshly formed 1-3 om atmospheric particles. The DEG-wCPC may be selected for its small size, ability operate in any orientation, and/or ability to measure 1 om particle. The DEG-wCPC has a power draw of 16 watts and is 18.5 cm by 16.5 cm by 30 cm. The DEG-wCPC may be calibrated using a high resolution half mini differential mobility analyzer having positive ammonium sulfate ion particles. The 50% cut point (ds0) for the DEG-wCPC may be 1.6 nm mobility diameter or 1.3 nm geometric diameter (see below). For the SA-RCPC model, the DEG CPC may be estimated to measure N4 and N_{≥4} with 100% efficiency and measure N3 with 30% efficiency.

Alternatively, the DEG-wCPC may be replaced with any condensation particle counter with d50 proximate to the 1-nm diameter range, such as, for example, the versatile water condensation particle counter (vWCPC, available from TSI) or the diethylene glycol mixing condensation particle counter (available from Airmodus).

FIG. 1 shows the SAD-RCPC comprising a small flow reactor coupled to a diethylene glycol inlet attached to a MAGIC water condensation particle counter (DEG wCPC, Aerosol Dynamics Inc.). The flow reactor used in this study was composed of borosilicate glass with dimensions of 5 cm ID, 44 cm long, with a 10 cm cone at the entrance of the flow. The DEG CPC samples through curved, 1.6 mm ID×100 mm long stainless-steel tube along the centerline to minimize wall loss in the reactor.

The flow reactor may be operated in two flow configurations to change the SA detection range. The high SA dilution (HD) configuration may use 35 cm³ min⁻¹ of SA flow through the side port and 1.0 L min⁻¹ of DMA through the main port. Computational fluid dynamic (CFD) simulations show a factor of 25 dilution of the SA concentration by the DMA flow. The low SA dilution (LD) configuration may use 900 cm³ min⁻¹ of SA flow through the main port and 100 cm³ min⁻¹ of DMA through the side port to dilute the SA by a factor of 1.04.

For each configuration, the CFD simulations show that the nucleation time may be estimated to be 25 s. The operation and dimension of the nucleation flow reactor may be chosen to minimize length while maintaining repeatable flow for a constant nucleation time, and accordingly, a wide range of dimensions and flow rates may be used when the nucleation time is 5-25 seconds.

DMA vapor (1 ppbv) may be generated by flowing nitrogen over a DMA permeation tube. Operating the nucleation flow reactor with high excess DMA may circumvent the need to control the exact concentration of DMA in the reactor. In addition, high concentration of DMA may reduce/eliminate ammonia or other amines in the sampled gas from appreciably participating in SA-DMA nucleation. The relative humidity (RH) in the DMA flow may be also varied to explore effects of relative humidity (RH) on the performance of the SAD-RCPC. The temperature may be room temperature (from 20° C. to 24° C.). Colder or hotter temperatures may influence SA-DMA nucleation rates with higher rates at colder temperatures and equivalent SA concentrations.

SA concentrations ranging from 2×10⁵ to 3×10⁹ molecules cm⁻³ may be generated using two methods. First, SA vapor may be generated by passing particle-free nitrogen or compressed air over an SA permeation tube. The permeation tubes may be made using a microporous PTFE tubing and filled with concentration liquid SA (1 mL of 98.6-99.99% SA by volume). The tubes may be sealed and let equilibrate for a week before being used to generate SA vapor. SA vapor may be also produced by passing nitrogen over a temperature-controlled liquid reservoir of SA. The SA entrained in nitrogen may be mixed with dry and humidified nitrogen in a large flow reactor with dimensions of 1.2 m×5 cm inner diameter. The SA concentration may be varied by changing the flow over the SA reservoir and dilution flow while maintaining a constant total flow.

The DEG-wCPC may be selected for its small size (18.5 cm×16.5 cm×30 cm), capability to operate in any orientation, and lower power draw (16 W). The DEG wCPC may be used to detect freshly formed 1-3 nm atmospheric particle. The DEG wCPC samples at 0.3 L min⁻¹ from the flow reactor. The temperatures for the DEG wCPC may be 60° C., 30° C., 10° C., 40° C., and 1° C. for the DEG0, DEG1, initiator, conditioner, and moderator stages respectively. The optics head may be maintained at 45° C. to reduce/eliminate condensation of DEG. The DEG wCPC may be calibrated using a positively charged, furnace-generated ammonium sulfate particles that may be size-selected with high resolution half mini differential mobility analyzer. The 50% cut point (d50) for the DEG CPC may be measured at 1.6 nm mobility diameter or 1.3 nm geometric diameter.

The sampled SA concentration by the SAD-RCPC (i.e., concentration at time equal to 0 s) may be calculated by iteratively solving the differential cluster balance equations (R7)-(R13) described below at the given nucleation time. The DMA concentration may be set to 1×10¹⁰ molecules cm⁻³ (1 ppbv). Varying DMA concentration by a factor of 0.1 to 10 may have negligible effects on calculated SA concentration. The initial SA concentration may be varied until the modeled output particle concentration ([N_{≥4}]) is within 0.01% of the measured particle concentration of the 1-nm CPC.

The SAD-RCPC may be configured to provide continuous particle and sulfuric acid concentration measurements with a time resolution of 1-2 Hz. This fast time response may make the instrument suitable not only for stationary measurements but also for moving platforms, such as aboard vehicles that are used for mapping community-wide aerosol concentrations. In addition, this instrument may be used to characterize rapidly changing aerosols, such as aerosols near sources in which concentrations of sulfuric acids may be highest due to minimal air dilution at point of emissions. This may be useful for industrial settings where a leak of sulfuric acid contamination may be tracked to its source.

The SAD-RCPC may have similar or better accuracy relative to the CIMS (factor of 2) with known background particle concentration. Depending on the background particle concentration, SAD-RCPC may detect sulfuric acid concentrations of at least 10⁵ molecules cm⁻³, which may be similar to CIMS. In addition, the SAD-RCPC's response may be stable across a wide range of relative humidities (e.g., 0-100%), temperatures (e.g., 270-320 K) and contamination compounds (e.g., oxalic acid, formic acid, and other volatile organic compounds, and low volatility organic compounds like those emitted from pump oils and engine exhaust) in the sampled air stream. This is in direct contrast with a conventional CIMS which has variable response due to uncertainties in chemical ionization under different operating conditions as described below.

Conventional CIMS used for real-time measurements may be large, heavy, and complex. The detection unit may require a reagent, typically nitrate, which may not be readily available in the field. In contrast, the SAD-RCPC comprises a small detection system, combining a flow reactor (20 cm in length), a DEG saturator (8-10 cm) and a wCPC (16-20 cm). Dimethylamine in the flow reactor may be generated from a small volume (<1 mL) of dilute dimethylamine in a sealed source within the secondary container. This may reduce/eliminate potential leaks and minimizes danger to the operator and public. The components of the SAD-RCPC may be combined or run separately to provide a larger use of both the flow reactor and the wCPC. The SAD-RCPC may have an overall size similar to commercially available benchtop condensation particle counters and will offer the capability of running on an external battery.

Characterization of urban atmospheric aerosols may utilize a large number of monitoring stations to provide sufficient information to build a detailed database of the urban environment. The use of low-cost sensors, high-spatial resolution monitoring of reactive gases that influence aerosol properties may be desirable. However, these sensors may suffer from at least one of the following limitations: the results may not be accurate with significant drift on the measurement and interferences with other atmospheric compounds; and low-cost sensors may be only available for a small number of air pollutants. More accurate and distributed observations of reactive gases may be useful to researchers, epidemiologists, and policymakers to improve human risk assessment studies, increase the database of emission sources, and/or evaluate the effects of control technologies. Thus, systems and methods that provide reliable data and are deployable at selected locations within a small area, or onboard moving platforms, may be desirable to complement low-cost sensor networks. Such systems may be characterized by being compact, affordable, and robust with fast time resolution.

Current measurements of gaseous sulfuric acid may be conducted using a chemical ionization mass spectrometer (CIMS); at this time, it is believed that no low-cost sensors or even medium-cost instruments exist to measure this compound. A CIMS, although sensitive to low concentrations of SA and with fast response, may be challenging to deploy in the field due to its large size (from 55×40×80 cm to 62×48×150 cm), heavy weight (up to 107 kg), and high-power consumption. In addition, the cost of a CIMS is high ($300K to $500K) which prevents deploying these instruments in a distributed measurement network for high spatial resolution measurements. Operation of a CIMS also may require detailed technical knowledge for maintenance and data analysis. It is believed that these combined factors, among others, may have prevented CIMS from being deployed for long periods of time at multiple locations. Consequently, no high spatial and temporal resolution observational data set exists for sulfuric acid within the urban environment.

The present invention may be directed to measure urban sulfuric acid concentrations. SAD-RCPC may be operated with a second, 1-nm condensation particle counter (CPC) to measure particle concentrations in the sample gas. The one CPC may measure the concentration of outdoor background particles and the second CPC may measure the total concentration of particles of particles produced in the SAD-RCPC flow reactor and outdoor background particle concentration. The difference between the two CPC-detected concentration is the concentration of particles produced in the flow reactor of the SAD-RCPC.

The SAD-RCPC may be characterized by fast measurement. The SAD-RCPC may provide continuous particle and sulfuric acid concentration measurements with a time resolution of 1-2 Hz. This fast time response may make the instrument suitable not only for stationary measurements but also for moving platforms, such as aboard vehicles that are used for mapping community-wide aerosol concentrations. In addition, this instrument may be used to characterize rapidly changing aerosols such as found near sources, where concentration of sulfuric acids can be highest. The SAD-RCPC may be useful for industrial settings where leaks of sulfuric acid contamination can be tracked to its source.

The SAD-RCPC may be characterized by accurate and sensitive measurement. The SAD-RCPC may have similar or better accuracy to that of the CIMS (factor of 2) with known background particle concentration. Depending on the background particle concentration, SAD-RCPC may detect sulfuric acid concentrations down to 10⁵ molecules cm⁻³, similar to CIMS. In addition, the instrument's response may be stable across a wide range of relative humidities, temperatures, and contamination compounds in the sampled air stream. This is in direct contrast with a CIMS having variable response due to uncertainties in chemical ionization under different operating conditions.

The SAD-RCPC may be characterized by comprising a compact, integrated, and safe instrument. Conventional CIMS used for real-time measurements may be large, heavy, and complex. The detection unit may require a reagent, e.g., a nitrate, which may not be readily available in the field. In contrast, the SAD-RCPC may comprise a small detection system, combining a flow reactor (20 cm in length), a DEG saturator (8-10 cm) and a wCPC (16-20 cm). Dimethylamine in the flow reactor may be generated from a small volume (<1 mL) of dilute dimethylamine in a sealed source within the secondary container. This may reduce/eliminate potential leaks and minimizes danger to the operator and public. The components of the SAD-RCPC may be combined or run separately to provide a larger use of both the flow reactor and the wCPC. The SAD-RCPC once packaged may have an overall size similar to commercially available benchtop condensation particle counters and maybe configured to run on an external battery.

The SAD-RCPC may be characterized by a more affordable cost relative to conventional instruments as well as maintenance and operating cost.

A method of using the SAD-RCPC may comprise atmospheric and aerosol research. A method of using the SAD-RCPC may comprise detecting and/or identifying principal contributors to the heterogencity of urban aerosol. A method of using the SAD-RCPC may comprise analyzing cloud formation related to new particle formation events, driven by chemical precursors, such as sulfuric acid. A method of using the SAD-RCPC may comprise analyzing aerosol-cloud interactions.

A method of using the SAD-RCPC may comprise indoor air quality research. For example, concentrated sulfuric acid is commonly used in the United States as a drain and toilet bowl cleaner. Gaseous sulfuric acid is emitted when these cleaners mix are used and likely drive rapid new particle formation indoors. This poses a unique and dangerous exposure risk to people using these cleaners and nearby bystanders as gaseous sulfuric acid and ultrafine particles may penetrate deep into the lungs and result in negative health outcomes. A method of using the SAD-RCPC may comprise monitoring air quality in areas having concentrated sulfuric acid, such as public bathrooms, schools, hotels, commercial offices, to protect the health of workers and public.

A method of using the SAD-RCPC may comprise analyzing industrial processes. Sulfuric acid is a key substance in the chemical industry, where it is commonly used in fertilizer manufacture. Other industries using SA include mineral processing, oil refining, wastewater processing, and chemical synthesis. Sulfuric acid is also used in large quantities by the iron and steelmaking industry to remove oxidation, rust, and scaling. Monitoring unwanted releases and ambient concentrations may help identifying leaks in the manufacturing processes as well as safeguard workers for undetected exposure to sulfuric acid vapors.

### EXAMPLES

The devices and systems according to the present invention, as well as methods of making and using the same, described herein may be better understood when read in conjunction with the following representative examples. The following examples are included for purposes of illustration and not limitation.

### Example 1

Experimental comparison of SAD-RCPC and CIMS

A custom-built transverse inlet, atmospheric pressure chemical ionization time of flight mass spectrometer (CIMS) is used as a comparison measurement to SAD-RCPC according to the present invention for the SA concentration in the sampled gas. The CIMS uses acetate (CH3COO—, CH3COOH·CH3COO—, H2O·CH3COO—) as the reagent ion to ionize SA vapor. The conversion of SA mass spectrometer signal to concentration has been previously explained with the ionization rate constant recently measured at 4.6×10⁹ cm³ s⁻¹ for acetate. SA generates by the flow reactor is simultaneously measured by the CIMS and SAD-RCPC. SA produced via permeation tube is analyzed by the CIMS before and after injection into the SAD-RCPC to account for any temporal changes in the permeation rate. Note, permeation tube injection of SA into the CIMS inlet results in factor of 16 dilution as modeled using CFD. The RH in the CIMS inlet and SAD-RCPC are also varied. For higher RH >60%, the RH in the CIMS inlet is kept at 60% to prevent loss of reagent ion signal. A wide range of sulfuric acid concentration is generated using sulfuric acid permeation tubes made of microporous PTFE tubing. The permeation tubes are scaled with glass plugs or heat pressed closed. Different carrier gases including nitrogen and varying cleanliness compressed air is used to deliver sulfuric acid vapor to the SA-RCPC and CIMS. The instruments are operated at room temperature between 21-25° C.

### Results and Discussion

FIG. 2A and FIG. 2B show the particle concentrations measured by SAD-RCPC compared to the SA concentration measured by the CIMS for the HD and LD configurations of the SAD-RCPC. For HD, SA rapidly dilutes after injection in the SAD-RCPC flow reactor, so less particles form at the same SA concentration when compared to LD. FIGS. 2A and 2B show that particle concentrations increase by the square of the SA concentration. This observation is in line with SA forming particles at the collision-controlled limit. In addition, the particle-free compressed air in the HD configuration results in fewer particles formed than the nitrogen measurements; however, this trend may not be observed with LD flows.

Converting measured particle concentrations into SA concentration relates to the DEG wCPC detection efficiency. The measured detection efficiency curve of the DEG wCPC is challenging to apply as the true detection efficiency of electrically neutral clusters composed of SA and DMA is not known. The DEG wCPC has a measured d50 of 1.6 nm mobility diameter with a detection efficiency of 9% at 1.3 nm mobility diameter (approximately the size of N4). However, as shown below, the predicted cluster concentrations of N4 from nucleation reactions of SA and DMA at SA concentration of 1×10⁷ molecules cm⁻³ is below 1 particle cm⁻³. In contrast, the DEG wCPC detects about 100 particles cm⁻³ in the LD configuration. This may suggest that the measured detection efficiency curve (based on positively charged ammonium sulfate particles) does not represent neutral SA-DMA clusters and/or the DEG has a non-zero detection efficiency of sub-1 nm clusters (N<4), which may significantly contribute to the total detected particle concentration.

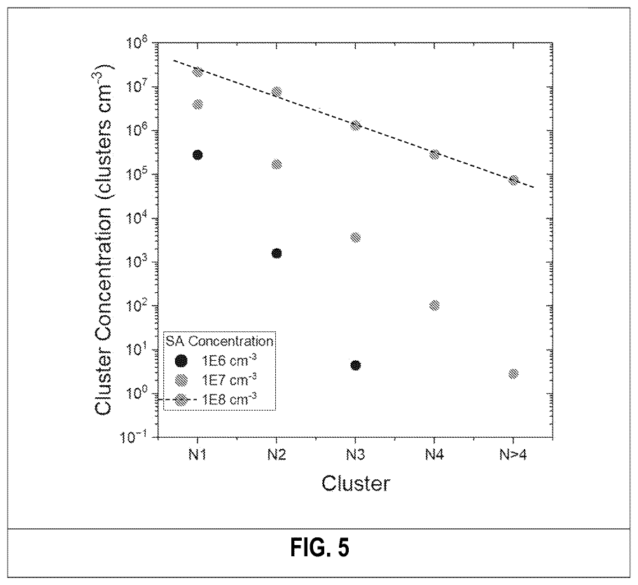

Since it is challenging to differentiate between these two possibilities, for the current conversion of particle concentration to SA concentration, a detection efficiency of 100% for N>4, and 30% for N3 is used. The DEG CPC may be able to detect a fraction of N2 which would increase the estimated SA concentration in the lower SA concentration ranges (<10⁷ molecules cm⁻³). This may be due to a higher fraction of the cluster population existing as N2 at lower SA concentrations (see FIG. 5). Sensitivity experiments are performed such that N2 concentration is detected between 0% to 2.5%. Increasing N2 detection efficiency decreases SAD-RCPC SA concentrations by on average 40% across the SA concentration range. Larger effects are observed on measurements with lower particle or SA concentrations as the ratio of N2/N>2 is larger at lower SA concentrations. Though the detection efficiency of N2 is not known, the good agreement between SAD-RCPC and CIMS assuming 100% for N≥4, and 30% for N3 detection efficiency seems to be sufficient to convert measured particle concentrations to SA concentration in the SAD-RCPC.

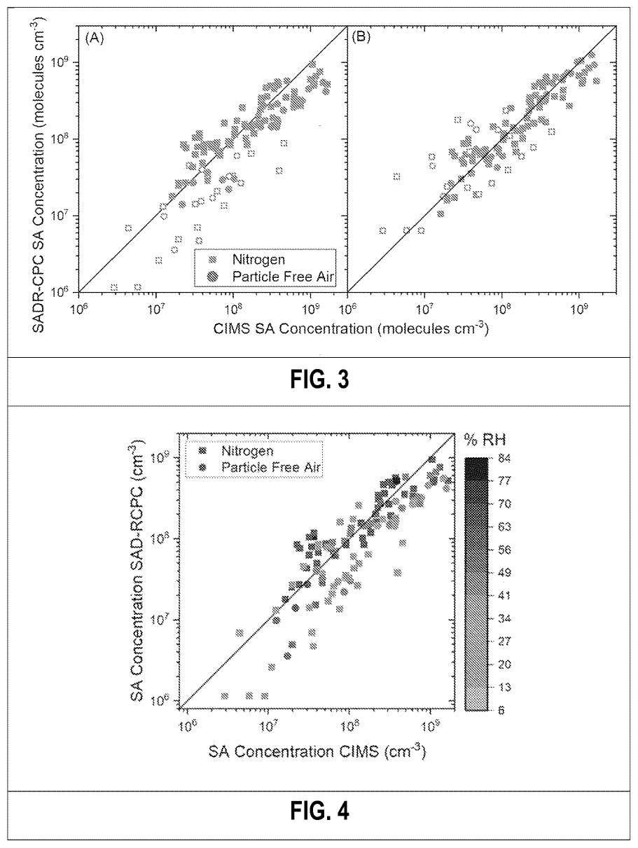

Converting measured particle concentration into SA concentration (Reaction 1) for the SAD-RCPC are compared to CIMS measurements in FIGS. 3A and 3B for both flow configurations. The HD configuration is able to detect SA concentrations ranging from 10⁷-10⁹ molecules cm⁻³. The LD detection range of the SAD-RCPC is from 2×10⁶ to 5×10⁸ molecules cm⁻³. At high SA concentrations (>3×10⁸ molecules for HD and >3×10⁸ molecules cm⁻³ for LD), the SAD-RCPC may underestimate SA concentration likely due to the increased coagulation rate. The model (Reaction 1) does not accurately capture the coagulation of larger particles (N>8) which results in overestimating particle concentrations at a given high SA concentration. The SA concentration at which this occurs depends on the dilution at of the SAD-RCPC. To measure above 3×10⁸ molecules, the HD DMA flow may be increased to increase dilution and reduce coagulation. Overall, the SAD-RCPC generally measures concentrations of SA in nitrogen higher than the CIMS with a mean relative error of 119%.

In contrast to nitrogen as the carrier gas, SAD-RCPC measurements of SA using particle-free compressed room air are about 30% below the CIMS. Compressed room air is observed to contain pptv-levels of dimethyl and trimethylamine along with volatile organic compounds which are likely present but not identified and/or detected by the acetate CIMS. These impurities in the air may be suppressing SA-nucleation to some extent which may lower the SAD-RCPC estimated SA concentration. More likely, the poorer agreement of the SAD-RCPC with the CIMS for room air may be due to compounds in room air altering the acetate ionization reactions. The acetate reagent ion signal is observed to increase by a factor of two which is unusual as this signal is typically constant for the SA concentrations discussed here. Numerous scenarios may cause this during these experiments increase in reagent ion signal. For example, unknown compounds attach to the acetate ions and enhance ionization rate constant with sulfuric acid. Another possibility is room air compounds coated the inlet and influenced the focusing of the atmospheric pressure ion cloud. Consequently, the uncertainty is relatively high for the CIMS when measuring SA in room air, which suggests adding additional ionization reactions are occurring and adds additional uncertainty to the CIMS compressed air measurements taken when the SAD-RCPC was in the HD configuration. The SAD-RCPC LD measurements are taken with a nitrate reagent on the CIMS, which do not interact with the compounds in the room air.

The SA-DMA nucleation model may be used to convert particle concentrations into SA concentrations when the reaction time and flow dilution in the reactor are known. In addition, an assumed detection efficiency of the DEG wCPC may be used. These factors may be avoided by simply correlating particle concentrations with CIMS measured SA concentration. This approach is shown in FIG. 3B with details on the fit n parameters fitting the statistical model.

The correlation assumes the CIMS concentration is accurate which is challenging to evaluate as numerous factors may influence CIMS accuracy as discussed above. In addition, when paired with a CIMS, the SAD-RCPC particle concentrations may be converted to SA concentration using a statistical model. The statistical regression model uses sufficient data to train and may need to be redone when conditions are changed. See Table S1.

To conduct atmospheric measurement of SA using the SAD-RCPC, it may be useful to understand how pre-existing particles influence the measurement. Particles in the sample stream may (1) reduce the SA-DMA formation rate of 1-nm particles due to coagulation losses and (2) obscure small changes in particle concentrations from SA-DMA nucleation reactions The coagulation sink of pre-existing particles may be quantified from Fuch's surface area and incorporated into the cluster balance equations. Higher coagulation sink may increase the lower detection limit of the SAD-RCPC. However, this is a minor challenge compared to detecting the contribution of SA-DMA nucleated particles (on the order of 1-1000 particles cm⁻³) formed in the SAD-RCPC over the background concentration of particles. In more polluted areas where the SA concentration may be within the range of the SAD-RCPC, typical background concentrations range from 10³ to 10⁴ particles cm⁻³. Pulse height analysis of DEG wCPC may be used to separate the SA-DMA particles, which may be <3 nm, from the background particles. This has successfully been used to quantify freshly formed particles in the upper boundary layer. Another option may be to remove background particles without influencing the SA concentration. This may not be done by filtration if this would severely reduce SA concentrations.

### Relative Humidity

To determine the effects of relative humidity on the SAD-RCPC, experiments with RH varying 6% to 84% are conducted with the SAD-RCPC and CIMS. The humidity is controlled by passing a carrier gas flow over a reservoir of HPLC grade water. The relative humidity of the CIMS measurements is controlled using the same technique. For measurements greater than 60% RH, the CIMS is kept at 60% RH to reduce/prevent signal loss. The results of the varied humidity experiments are shown in FIG. 4. At higher RH, the SAD-RCPC measures higher SA concentrations than at corresponding low RH conditions. This variation it is small compared to the overall variation of the measurements. An RH dependence may be added to the SAD-RCPC model to account for the weak dependence.

### Reaction Model Equations for SAD-RCPC Model

The reaction model for the SAD-RCPC, (R7)-(R13) is derived from (R1)-(R6). The reaction model may be solved using a numerical method for ordinary differential equations. For example, the model may be solved using odeint in Python.

$$\frac{dSA}{dt} = - {k}_{f} [ SA ] [ B ] - q [ SA ] - μ [ SA ] \qquad ( R 7 )$$

$$\frac{dDMA}{dt} = - {k}_{f} [ SA ] [ DMA ] - q [ DMA ] - μ [ DMA ] \qquad ( R 8 )$$

$$\frac{{dN}_{1}}{dt} = {k}_{f} [ SA ] [ DMA ] - {k}_{f} [ {N}_{1} ] \times ( 2 [ {N}_{1} ] + [ {N}_{2} ] + [ {N}_{3} ] + [ {N}_{4} ] + [ {N}_{> 4} ] ) - q [ {N}_{1} ] - μ [ {N}_{1} ] \qquad ( R 9 )$$

$$\frac{{dN}_{3}}{dt} = {k}_{f} [ {N}_{1} ] [ {N}_{2} ] - {k}_{f} [ {N}_{3} ] \times ( [ {N}_{1} ] + [ {N}_{2} ] + 2 [ {N}_{3} ] + [ {N}_{4} ] + [ {N}_{> 4} ] ) - q [ {N}_{3} ] - μ [ {N}_{3} ] \qquad ( R 10 )$$

$$\frac{{dN}_{4}}{dt} = {k}_{f} [ {N}_{1} ] [ {N}_{2} ] + {k}_{f} [ {N}_{2} ] [ {N}_{2} ] - {k}_{f} [ {N}_{4} ] \times ( [ {N}_{1} ] + [ {N}_{2} ] + [ {N}_{3} ] + 2 [ {N}_{4} ] + [ {N}_{> 4} ] ) - q [ {N}_{4} ] - μ [ {N}_{4} ] \qquad ( R 11 )$$

$$\frac{{dN}_{> 4}}{dt} = {k}_{f} [ {N}_{4} ] \times ( [ {N}_{1} ] + [ {N}_{2} ] + [ {N}_{3} ] + [ {N}_{4} ] + [ {N}_{> 4} ] ) + {k}_{f} [ {N}_{3} ] \times ( [ {N}_{2} ] + [ {N}_{3} ] + [ {N}_{> 4} ] ) - {{k}_{f} [ {N}_{> 4} ]}^{2} - q [ {N}_{> 4} ] - μ [ {N}_{> 4} ] \qquad ( R 12 )$$

$$\frac{d μ}{dt} = - {μ}^{2} \qquad ( R 13 )$$

The collision rate, kf, of 4.2×10⁻¹⁰ cm³ s⁻¹ is assumed to be equal for all clusters. The wall loss rate, q, is assumed to be diffusion-limited and calculated using the equation q=3.66D/r where D is the diffusion coefficient of the cluster in air at STP and r is the radius of the flow reactor. A wall loss rate is calculated for each cluster and ranges from 0.05 s⁻¹ for SA to 0.045 s⁻¹ for N≥4. The dilution rate, μ, is a function of time and is calculated using (R13). The squared dependence of the dilution rate is calculated empirically by matching the equation to the dilution calculated by CFD. For the HD and LD configurations, μ at t=0 is 1 s⁻¹ and 0.004 s⁻¹, respectively, using the CFD simulation.

### Cluster Size Dependency on SAD-RCPC

The detection efficiency of the DEGCPC for each of the SA-DMA clusters may be a large source of uncertainty for the SAD-RCPC. To demonstrate this, concentrations of each cluster type (N1-N>4) are simulated using the reaction model discussed above at different SA concentrations for the LD configuration. These concentrations are shown in FIG. 5.

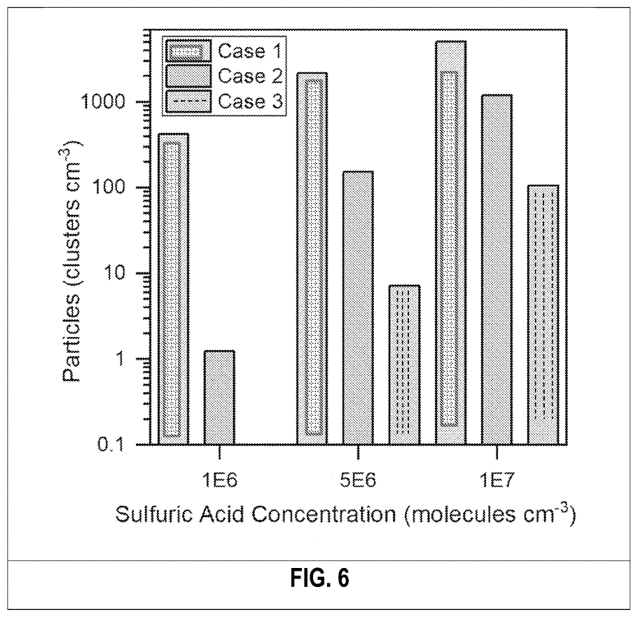

FIG. 6 shows the predicted measured particle concentration measured by the DEGCPC at three different simulated sulfuric acid concentrations. Case 1 assumes detection efficiencies of 0.1% N2, 30% N3, and 100% N≥4. Case 2 assumes detection efficiencies of 30% N3, and 100% N≥4. Case 3 assumes detection efficiencies of 100% N≥4. The predicted measured theoretical detected particle concentration varies the most at low concentrations of SA where the ratio of smaller clusters to larger clusters is much larger. The SAD-RCPC is tested with many different combinations of detection efficiencies to determine values that resulted in a good match between SAD-RCPC and CIMS measurements, which is determined to be Case 2.

The detection efficiency of the DEGCPC is measured using a nano-DMA and electrometer. The ammonium sulfate particles are generated using an atomizer and charged using a Po-210 source. The particles are size sorted into monodispersed flow using the nano-DMA. The monodispersed aerosols are then measured using the DEGCPC and electrometer to calculate the detection efficiency. The size of the aerosol particles is calculated from the voltage and flow of the nano-DMA. There are uncertainties with these measurements, the largest being the unknown effects of charge and composition on the detection efficiency.

### Statistical Model for Calculating SAD-RCPC Sulfuric Acid Concentration

The particle concentration measured by SAD-RCPC may be converted to SA concentration using an empirical fit based upon measured sulfuric acid concentration by the CIMS. FIG. 3 (B) displays the fitted SADR-CPC concentration using the following equation:

$${log}_{10} [ SA ] = m log ( [ Particles ] ) + log ( k ) .$$

The coefficients and score of the fits are displayed in Table 1 below. This fitted equation may be reevaluated with CIMS measurements if any portion of the SADR-CPC changes; this includes flow reactor dimensions, flow rates, DEG wCPC operating temperatures, or sampling rates.

| | m | k | R² |
|---|---|---|---|
| HD Nitrogen | 0.403 | 6.88 | 0.827 |
| HD Particle Free Air | 0.368 | 7.41 | 0.929 |
| LD Nitrogen + Particle Free | 0.305 | 6.90 | 0.475 |
| Air | | | |

### Conclusion

The Sulfuric Acid Dimethylamine Reactive Condensation Particle Counter (SAD-RCPC) may be capable of measuring atmospherically relevant concentrations of sulfuric acid from 1×10⁵ to 1×10⁹ molecules cm⁻³. The SAD-RCPC operates by reacting sulfuric acid with DMA to form detectable 1 nm particles. The SAD-RCPC comprises a compact flow reactor and a DEG wCPC. Two approaches are provided for determining SA concentration from measured particle concentrations for the SAD-RCPC. First, the nucleation SA-DMA reactions and fluid dynamics are modeled in the SAD-RCPC to determine the particle concentration at a given initial SA concentration. This approach also utilizes a fitted detection efficiency in the model to match modeled particle concentrations to DEGCPC measurements. Second, a statistical model fitted from CIMS SA measurements and SAD-RCPC particle measurements may be used. Both methods are valid but require different tools for validation. Ultimately, comparison of the SAD-RCPC with the traditionally used CIMS showed good agreement with a mean relative error of 73%.

The SADR-CPC may provide a lower cost, more portable, and less power-intense alternative to measuring atmospheric sulfuric acid vapor compared to the conventional used CIMS. The detection range includes the majority of atmospheric sulfuric acid concentrations previously observed by CIMS. The existence of preexisting particles in the sample gas may increase the lower detection limit depending on the coagulation sink rate and obscure the concentration of particles formed in the SAD-RCPC.

### Example 2

### Glass Reactor

A glass flow reactor for the sulfuric acid dimethylamine reactive condensation particle counter (SAD-RCPC) is shown in FIG. 8. The glass reactor uses perpendicular flows to rapidly mix the sample gas and dimethylamine reagent flow in the inlet cone. Increasing the mixing of the sample gas and reagent may improve reproducibility of measurements. The reaction time of the glass flow reactor may be controlled by changing the main flow rate or by changing the port that is used to sample the process flow. For low concentrations of sulfuric acid (2×10⁶ to 5×10⁸ molecules cm⁻³), the main flow is sample gas, and the side flow is dimethylamine to reduce dilution effects of the sample gas. At higher concentrations of sulfuric acid (10⁷ to 10⁹ molecules cm⁻³), the main flow may be switched to dimethylamine and the side flow of sample gas. This configuration may dilute the sample gas and prevents the condensation particle counter from over-ranging the condensation particle counter. Both configurations use dimethylamine concentrations in the ppbv range.

The glass flow reactor may comprise of three separate glass pieces of borosilicate having o-rings and clamps connecting the pieces. The glass is naturally inert and does not react with sample gas or reagents. The modular design allows the user to exchange different geometries of the reactor depending on the needs. The glass flow reactor may be useful for laboratory measurements due to the fragility of glass.

The inlet design of the glass flow reactor does not separate pre-existing particles from the sample flow. These background particles are subsequently measured by the CPC in addition to the particles formed in the flow reactor (via reaction of sulfuric acid with dimethylamine). At low sulfuric acid concentrations, the number of particles generated by nucleation reactions of sulfuric acid-dimethylamine may be smaller than the background particle concentrations.

### Aluminum Reactor

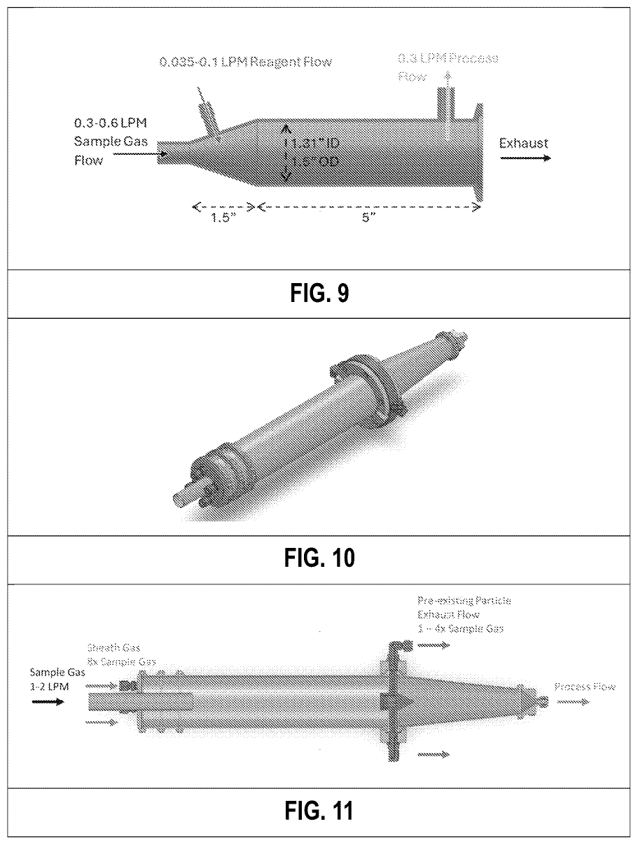

The aluminum reactor, shown in FIG. 9, may be configured to mix sample gas and reagent flow in the inlet cone, similar to the glass reactor discussed above. The aluminum reactor may comprise a unitary piece of aluminum including the inlets, reactor chamber, and outlets. Alternative, the reactors comprising the inlets, reactor chamber, and/or outlets may comprise separate pieces coupled together. The aluminum reactor comprises one port to sample process flow so the reaction time may only be controlled by changing the flow rates. The aluminum may be coated in either INERTIUM (available from AMCX, LLC) or SILCONERT (available from Silco Tek) to reduce/prevent sulfuric acid and/or other gases from reacting with the aluminum and forming oxides which may affect measurements.

The aluminum flow reactor is 5 inches from the bottom of the inlet cone to the end of the reactor. The reactor has an outer diameter of 1.5 inches and a wall thickness of 0.095 inches.

The aluminum flow reactor may be configured for field measurements. The flow reactor may be smaller, lighter, and more robust than glass. It may be light enough to be mounted on drones or tethered balloon systems to obtain vertically resolved measurements.

The flows within the aluminum flow reactor may be adjusted to varying the sulfuric acid-dimethylamine reaction time (i.e., residence time). Longer reaction time may convert more sulfuric acid into detectable particles. However, longer reaction time also translates to higher wall losses and scavenging by pre-existing particles. The reactor may be operated between 5-25 s reaction time.

### Core Flow Reactor

The core flow reactor design is shown in FIG. 10. The core flow reactor may be configured to separate pre-existing particles from particles created by the reactive gas nucleation by exploiting the reactive gas's faster diffusion than particles through air. A cross-sectional view of the reactor is shown in FIG. 11. In use, sample gas may enter the reactor through the large tube on the left. The clean sheath flow, mixed with dimethylamine, may enter through at least one of the four ports small ports on the left side of the reactor. The sheath flow may be laminarized using three metal screens before the sheath gas meets the sample flow. The sheath gas flow rate may be 1-10 times, such as eight times, the sample gas flow rate, the ratio of the cross-sectional area of the sheath and/or sample gas entrances, to minimize turbulence when the flows meet.

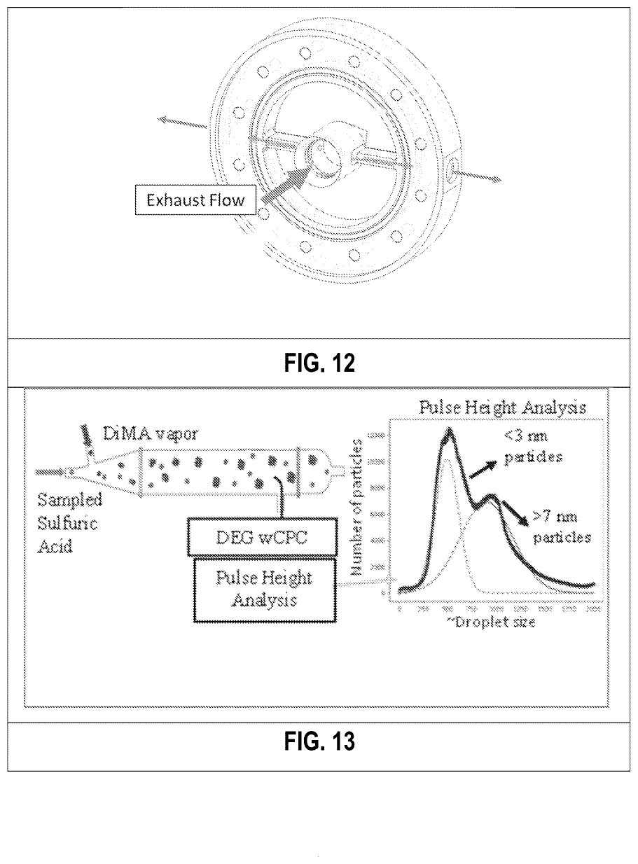

Within the body of the core flow reactor, the reactive gas (such as sulfuric acid) may diffuse radially outward into the sheath flow faster than the larger pre-existing particles and nucleate to form new 1-3 nm particles in the sheath flow. Sulfuric acid may have an estimated diffusion velocity of about 1 cm/s. Pre-existing particles may be removed from the reactor using the center flow extractor, shown in FIG. 12. The center flow extractor may pull the pre-existing particle flow through a sintered filter and then through two bores within the support beam. The sintered filter may be needed to ensure even flow across the diameter of the flow extractor. The extractor flow rate may be adjusted to eliminate the pre-existing particles. The remaining sheath flow containing the freshly nucleated particles may continue into the cone and may be sampled using a CPC.

The ability of the core flow reactor to remove pre-existing particles may allow this reactor to be used in outdoor environments where the pre-existing particle concentration is high enough to obscure the detected concentration of sulfuric acid-dimethylamine-formed particles. In addition, a high pre-existing particle population in a standard mixing reactor may act to scavenge and reduce the concentration of reactive gas and newly formed particles. Consequently, background particles limit the lower range of sulfuric acid the SAD-RCPC may detect. Removing background particles may allow the SAD-RCPC to detect lower concentrations of sulfuric acid and reduce the signal-to-noise ratio of the instrument. The core flow reactor may be optimized for weight, length, and reaction time depending on the targeted sulfuric acid concentration. Each configuration of the core flow reactor for the SAD-RCPC may be compared to a chemical ionization mass spectrometer to determine the relationship between detected particles and sulfuric acid concentration.

### Example 3

Gaseous sulfuric acid (SA, H₂SO₄) plays a key role in the heterogeneous distribution of urban atmospheric aerosol particle concentrations and composition. SA may be a main driving compound for atmospheric new particle formation (NPF), the process whereby gaseous precursors react to form stable aerosol particles. These particles grow to detectable sizes and may be an important source of ultrafine particles (i.e., particles <100 nm in diameter) and help contribute to their heterogeneous distribution across cities. For example, urban measurements have shown that SA-driven NPF may contribute up to 90% of the total aerosol number concentration during a NPF event. In addition, particles freshly formed in the urban center may be transported outside the city and increase the surrounding particle concentrations.

The rate at which new particles are formed in the atmosphere may heavily depend on the concentration of SA and/or pre-existing particles. Both quantities vary over short spatial and temporal scales in cities due to proximity to emission sources. SA may be formed in urban environments from photochemical reactions of gaseous precursors, specifically sulfur dioxide (SO2) which may be emitted from vehicle exhaust and other fossil fuel combustion sources. As traffic and solar radiation vary considerably in the urban environment, the concentrations of SA and its contribution to aerosol size distributions via NPF may also vary.

SA may also influence aerosol composition. Particulate SA, measured as sulfate (SO₄²⁻) by aerosol mass spectrometers, accounts for about 10-20% of particulate mass. Measurements in Oakland, CA, demonstrate that the sulfate mass varied by roughly a factor of five across the city with the highest concentrations near downtown, highways, and shipping ports. The variation in particulate sulfate concentrations may be due to proximity to fossil fuel combustion sources. SO₂ emitted from these sources may rapidly photo-oxidize in the hot exhaust into SA. The fraction of SA that remains in the gas phase may depend on the aerosol size distribution. Particles scavenge SA, which in turn may increase the particulate sulfate mass. As a result, the concentration of gaseous SA may relate to and influences the composition of urban aerosol particles.

As traffic, emission sources, solar radiation, and particle size distributions vary considerably throughout the urban environment, the SA concentrations may exhibit significant variations on small spatial and temporal scales. Consequently, hyperlocal events of NPF may occur that vary in intensity and frequency. Contributions of these NPF events to aerosol concentrations may by hidden in the diurnal variation and heterogenous distribution of ambient particle concentrations.

Currently, gaseous SA may be measured by a chemical ionization mass spectrometer (CIMS). Deployment of a CIMS at high spatial resolution may be challenging due to its large size (from 55×40×80 cm to 62×48×150 cm), heavy weight (up to 107 kg), and high-power intensity (2 kW). In addition, a CIMS may be prohibitively expensive ($300-500K) to deploy in a high spatial resolution measurement network. Operating a CIMS may require frequent maintenance due to complex electronics and uncertain chemical ionization processes. As a such, using CIMS requires detailed technical knowledge that takes years of training to develop. The data analysis may be arduous and complex with special experimental and programming skills needed to convert signals to sampled concentrations. These reasons combined have made deploying several CIMS for long periods of time at multiple locations extremely challenging. Consequently, no high spatial and temporal resolution observational data set exists for SA within the urban environment.

The key challenge with measuring SA is its low ambient concentrations. Urban concentrations of SA vary between 10⁵-10⁹ molecules cm⁻³. As a result, instruments need high sensitivity to accurately detect SA above background interfaces from other trace compounds. Though CIMS is powerful enough to measure SA, the instrument still suffers from significant uncertainties due to unknown parameters that impact chemical ionization processes in the inlet and ion transport through the instrument. The estimated uncertainty for CIMS is roughly a factor of 2.

The present invention is directed to a new technique to measure ambient SA. The technique, referred to as the sulfuric acid dimethylamine reactive condensation particle counter (SAD-RCPC), leverages the ability of SA to nucleate into detectable 1-nm particles with dimethylamine (DMA, DiMA) in a predictable manner. SAD-RCPC, as depicted in FIG. 1, comprises a compact nucleation flow reactor (20 cm long×3.8 cm OD) where >1 ppbv gaseous at least one of DMA, DiMA, and ((CH₃)₂NH) is mixed with sampled air. The vapors nucleate into 1 nm particles during a controlled amount of time between 10-25 s. Particles may be then counted with a diethylene glycol inlet coupled to a water condensation particle counter (DEG wCPC). Pulse height analysis (PHA) on the droplets produced in the DEG wCPC may be also deployed to distinguish >7 nm diameter background particles from the freshly nucleated SA particles formed in SAD-RCPC. The concentration of formed particles may be directly related to the SA concentration in the sampled air. As such, the concentration of nucleated particles may be used to back-calculate the sampled SA concentration following an acid-base nucleation reaction scheme discussed above. This reaction model is a series of rate laws that model the formation of a 1-nm particles from SA and DiMA. The model may have inputs of nucleation reaction time in the flow reactor, DiMA concentration, and concentration of nucleated particles and outputs SA concentration.

FIG. 3 shows good agreement between SAD-RCPC and the CIMS of lab-generated SA dispersed in particle free nitrogen or room air over a wide range of relative humidities (RH). SA may be generated by passing nitrogen gas over liquid sulfuric acid. These results demonstrate the feasibility of the SAD-RCPC for measuring SA. The accuracy of the SAD-RCPC may be similar to that of the CIMS (factor of 2) though the concentrations measured by the SAD-RCPC may be more repeatable than the CIMS. The horizontal variability seen in FIG. 3 is primarily the result of the CIMS reagent ion signal fluctuating due changes in RH and trace contamination influencing how nitrate reagent ions chemically ionize SA.

SAD-RCPC measurements of SA in Pittsburgh, PA has not produced comparable SA concentrations as observed by the CIMS. FIG. 19 displays PHA of Pittsburgh air. The high background particle concentration in Pittsburgh results in a very large >7 nm particle pulse height distribution. This large peak is obscuring a <3 nm peak produced from particles formed in the reactor. As a result, the estimated SA concentration from the SAD-RCPC may be too high in the urban environment.

A benchtop SAD-RCPC according to the present invention may be configured to measure SA in the urban atmosphere. The urban atmosphere may be different from measuring SA in a cleaner environment where the DEG wCPC's pulse height distribution of background particles is separable from the SA-DiMA particles.

A benchtop urban SAD-RCPC may comprise a SAD-RCPC adapted for the urban environment to accurately measure the background concentration of particles in addition to concentration of nucleated particles in the reactor. SAD-RCPC may comprise a of a nucleation flow reactor coupled to two, 1-nm condensation particle counters. The SAD-RCPC's flow reactor dimensions and residence time may be 20 cm long by 3.8 cm inner diameter and 10-25 s. One CPC may measure ambient particle concentration and the second may measure the combined concentration of SA-nucleated particles in the flow reactor and background particles. The concentration difference between the two CPC may represent the number of particles generated in the nucleation reactor which then may be converted to SA concentration following the acid-base nucleation model (more details below). The dual CPC technique may circumvent the error in PHA due to high background particle concentrations.

The SAD-RCPC may be characterized as a compact, robust, and battery-operated instrument. The SAD-RCPC may comprise a flow reactor and a two DEG-wCPCs to provide real-time measurements of ambient SA with similar accuracy to the more cumbersome, complicated, power-intense CIMS. The small size, light weight, and straightforward control of the SAD-RCPC may allow users to deploy the system in different environments and field campaigns and run unattended for long periods of time. The cost of the system, an order of magnitude lower than the current CIMS, may broaden the number of users, as well as the number of units that may be deployed for a more extensive network. With a larger number of monitoring stations, researchers may better understand the heterogenous distribution of atmospheric aerosols in the urban environment by linking emission sources and atmospheric processes that result in new particle formation events.

Industries, government monitoring facilities, and academic research laboratories require gaseous SA information but have been unable to conduct these measurements due to the expense and complexity of operating a CIMS. In the industrial setting, trace SA is a common semiconductor containment during the manufacturing process. Government facilities, such as those run by DOE ARM, EPA, and NSF, which monitor atmospheric pollutants may benefit from the SAD-RCPC as SA plays a key role in atmospheric aerosol formation and aerosol composition. In addition, the SAD-RCPC may enable government research teams to deploy a long-term monitoring network of SA in diverse environments. In academia, numerous research labs conduct secondary aerosol formation chamber experiments but are unable to constrain the contribution of SA from organics on particle formation and growth due to lack of SA measurements.

The SAD-RCPC may comprise a benchtop flow reactor with two CPCs with counting capability down to 1 nm. Water-based condensation particle counters (wCPC) have been used to measure aerosol particle number concentrations for many decades. These counters use supersaturated environments to activating nanometer-size particles and further growing them to optically detectable droplets in the micrometer size range. Scattered radiation of a laser beam may be used to count single particles.

The system according to the present invention may comprise a DEG saturator coupled to a three-stage, water-based condensation particle counter. The DEG inlet may increase the range of detection of the wCPC while minimizing the chemical dependency and/or different responses to positive and negative charged particles. The DEG wCPC includes a two-stage DEG saturator built of aluminum blocks perforated to be crossed by a tubular wick. The blocks may be separated by an insulator and heated with independently controlled cartridge heaters. Using two stages for the DEG saturator may reduce the length required to achieve maximum saturation thus minimizing diffusion losses.

The first stage of the wCPC may be configured as the second stage (the condenser) of a DEG-butanol CPC for a simpler compact instrument. The DEG saturator may be connected to the inlet of the wCPC and controlled using the controller board of the main unit. The regions of DEG and water supersaturation may overlap to lower the smallest size detectable and minimize reduce the particle chemical dependance.

The DEG wCPC system according to the present invention may be characterized by at least one of: the DEG wick may need to be wetted each 1-3 days; the DEG may condense in the cold section of the wCPC (conditioner) such that the water wick needs to be wetted each 1-3 days; the attachment of the DEG saturator to the wCPC may or may not be temperature controlled; the attachment of the DEG saturator to the wCPC may comprise metal walls. The supersaturation profile may be improved by having a closer connection between the DEG saturator and wCPC.

The wCPC system according to the present invention comprises a single growth tube channel with a flow rate of 300 cm³ min⁻¹. The length and temperatures of each stage may achieve particle growth of at least 4 nm. The particle counter measures 40 mm×40 mm×110 mm and operates at an air sampling flow of 0.3 L min⁻¹, with a Dₚ50=5 nm at a temperature difference of 35° C. The dynamic range for accurate counting may vary from 0.1 to 3×10⁵ particles cm⁻³, which may be sufficient to detect ambient nanoparticle formation events. The wCPC system may comprise a dual channel counter, such as a three-stage unit (MAGIC®), using common stages for both growth tubes with a single controller managing the temperature of each stage. The wCPC system may comprise a single heatsink for heat release. The wCPC system may comprise a dual heatsink for heat release.

The D2CPC may detect and measure aerosol and ambient particles. For example, the D2CPC may detect ammonium sulfate particles having diameters between 8 and 100 nm. A wire generator is used to generate particles between 3 nm and 8 nm. Particles smaller than 3 nm are generated using an electrospray and a high-resolution half-mini differential mobility analyzer (DMA). Using this technique, particles of different chemical composition and charge polarity are generated to determine the sensitivity of the D2CPC. The bench top D2CPC may be characterized by a 50% detection efficiency of 1.5 nm mobility diameter, and <5% reading accuracy between channels for particle diameters between 1.5 nm and 10 nm.

The system according to the present invention may comprise a D2CPC and a SAD-R flow reactor for continuous monitoring of the generated particles. The SAD-R comprises an aluminum flow reactor having a length of 20 cm and an inner diameter of 3.8 cm (see FIGS. 9-11). Aluminum may minimize the weight of the reactor while maintaining its robustness. To minimize wall losses of polar gases onto the oxidize aluminum, the reactor may be passivated with INERTIUM. Sample flow may enter through the top cone at 1 L min⁻¹ and mix with >1 ppbv DMA at 100 cm³ min⁻¹. The nucleation reaction time may be 10 s with these flow rates measured by tracking the time at point of mixing to the sampling inlet of the DEG wCPC. Varying the sampling flow rate may change the nucleation reaction time.

The SA-DMA nucleation model includes a series of second order reactions that convert SA into a 1-nm particle at the gas collision rate. This particle may include four SA and four DiMA molecules. The reaction rate laws (i.e., cluster balance equations) may be iteratively solved by changing the initial SA concentration until the modeled 1-nm particle concentration after the 10 s nucleation time matches within 0.01% of the measured concentration of freshly formed particles. Nucleation of SA with DiMA produces a distribution of particle sizes of about 1 nm. The CPC may detect 1-nm particles at less than 100% efficiency but have sufficient efficiency to count clusters of larger size. As a result, the contribution of the >1 nm particles may balance out the undercounted smaller clusters to the total concentration when analyzed by the SA-DiMA models that only account for 1-nm particles. FIG. 2 shows these errors may cancel out and laboratory results are in good agreement between the SAD-RCPC and the CIMS.

Referring to FIG. 6, a sensitivity analysis on the SA-DiMA model show certain parameters may influence estimated SA concentration from measured 1-nm particle concentrations. The model may be insensitive to changes in DiMA concentration when its concentration is 10 times greater than the SA concentration. The permeation rate of DiMA may be >1 ppbv (10⁹ molecules cm⁻³) in the SAD-R, so a DiMA concentration of 1 ppbv may be used in the model regardless of its actual concentration. Furthermore, the estimated SA concentration may be sensitive to the coagulation rate (i.e., concentration of pre-existing particles) with higher rates reducing the number of SA-DiMA particles produced. To reduce the effects of coagulation loss, shorter nucleation times in the SAD-R may be used by increasing the flow rate through the reactor.

The SAD-RCPC that operates with two CPCs may use one CPC to measure background particles flow reactor and the second at the sampling port after SA reacts with DiMA. The connections lengths may be minimized to reduce sampling wall losses and equal in length to ensure identical wall losses. Known concentration of 2, 3, 10, and 20 nm aerosol particles may be separately generated via furnace or atomizer and size selected by a DMA (either high resolution half-mini or a TSI nano). Size-selected particles may be introduced into the SAD-R2CPC at 1 L min⁻¹ with 100 cm³ min⁻³ side flow without DiMA to mimic the typical operation of the SAD-R. The difference in particle concentrations of the D2CPC may provide the wall loss fraction which may be used to correct the second channel of the D2CPC for losses of background particles in the SAD-R. Temporal trends on particle loses, if any, may be determined over time, as walls get coated with particles.

For ambient sampling, the SAD-RCPC's flow reactor may utilize a single pump to pull about 1 L min⁻¹ of air into the reactor. The exhaust of the SAD-RCPC and pump may be then passed through a filter and activated carbon, with 100 cm³ min⁻¹ of this flow directed over the DiMA permeation tube to generate DiMA vapor that may be then injected into the SAD-R. The pump may be powered and controlled by a variable 12 V. The pump operation may be integrated to the CPC controller board such that the flow rate through the flow reactor may be actively monitored and/or maintained.

When a large wall loss fraction indicates that the SAD-RCPC reactor may significantly reduce the number of particles observed by the CPC measuring flow reactor and background particles), the SAD-RCPC flow rates may be adjusted to minimize turbulent mixing wall losses of the 2-10 nm particles which occurs in the entrance cone (see FIG. 3). The dimensions of the SAD-RCPC may also be modified by enlarging the inner diameter of the tube to also reduce wall losses. A second, identical flow reactor without added DiMA may also be connected to the background particle CPC to simulate equivalent wall loss due to the SAD-RCPC between the two CPCs. Thus, the particle difference between the 2 CPCs may be solely due to particles generated in the SAD-RCPC.

The performance of SAD-RCPC may be compared to its performance with concurrent measurements of a CIMS reference unit. The standard method for measuring sulfuric acid is a CIMS. Referring to FIG. 1, two methods for generating repeatable and tunable concentrations of SA vapor in particle-free carrier gas may be used.

FIG. 13 includes a schematic of the SAD-RCPC comprising a nucleation flow reactor shown in FIG. 1 coupled to a DEG wCPC and a pulse height analysis (shown in the right graph) of Pittsburgh, PA air demonstrating the separation and detection of particles formed in the flow reactor from ambient particles.

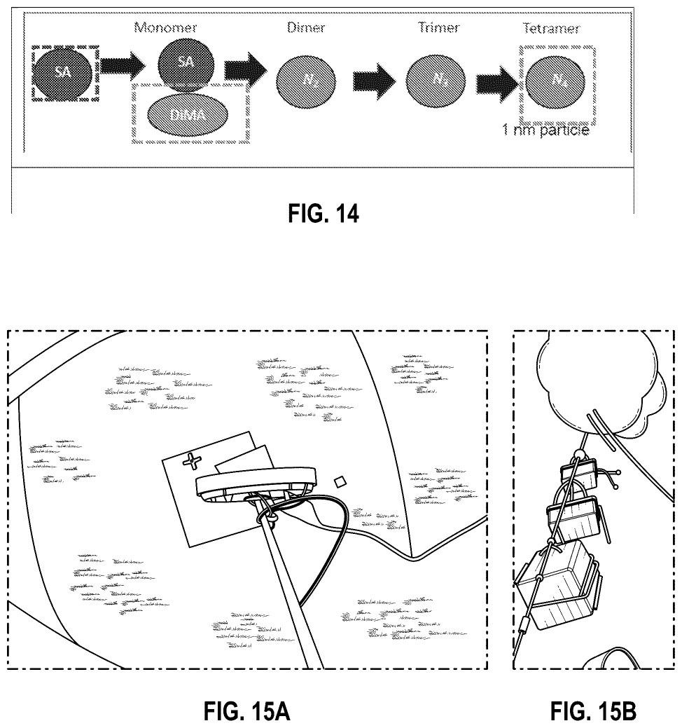

FIG. 14 includes a schematic of an acid-base reaction model of SA monomer (SA) nucleating with dimethylamine (DiMA) to form a dimer (N2), a trimer (N3), and a tetramer (N4) which is a 1 nm particle. The inputs to the model are the nucleation time, 1-nm particle concentration, and dimethylamine concentration (boxed DiMA). The model outputs sampled SA concentration (boxed SA).

FIG. 15A and FIG. 15B include a deployable TBS-mounted DEG-wCPCs according to the present invention to measure an effective base concentration ([B_{eff}]) and sulfuric acid (SA) concentration.

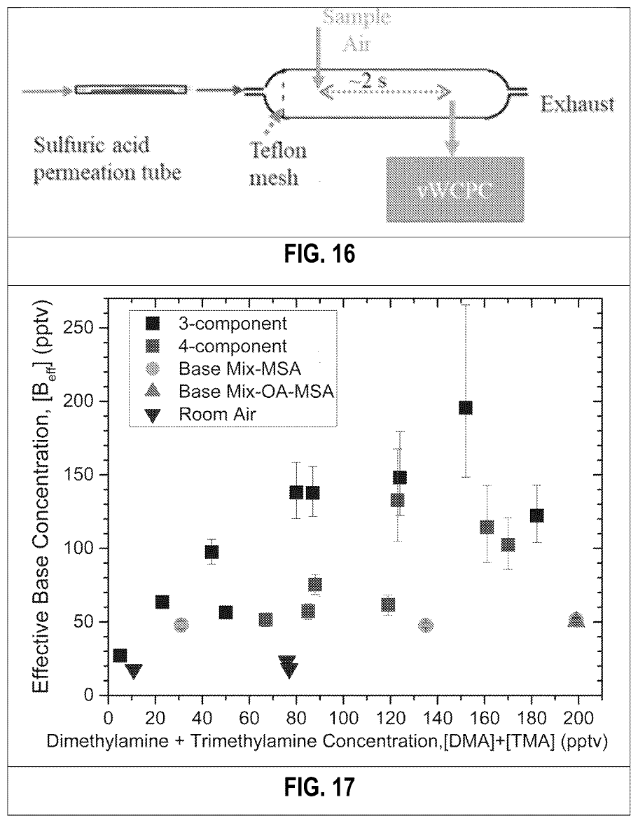

FIG. 16 includes a schematic of a base-CPC according to the present invention to measure the effective base concentration, [B_{eff}]. The base-CPC comprises a sulfuric acid flow reactor and a 1 nm versatile water condensation particle counter (vWCPC). Any 1-nm CPC may be used instead of the vWCPC such as the DEG-wCPC. Teflon mesh is used to straighten the flow but is not required if sulfuric acid-base nucleation reaction times are known.

FIG. 17 includes a chart showing individual and combinations of stabilizing compounds sampled by the base-CPC, and the resulting [B_{eff}] as a function of measured base concentration. The mixtures include ammonia, methylamine, dimethylamine, trimethylamine (with this mixture called Base Mix), methanesulfonic acid (MSA), oxal acid, formic acid, acetic acid (with this mixture called OA), and particle-free room air.

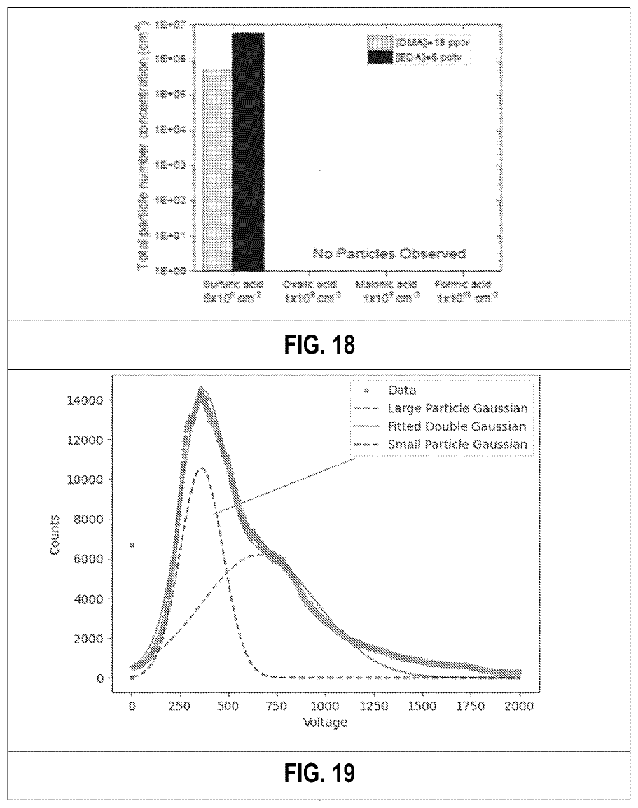

FIG. 18 includes a chart showing total observed particle concentration larger than 1 nm produced from reactions of sulfuric, oxalic, malonic, or formic acid and dimethylamine (DMA) or ethylene diamine (EDA). This chart shows that dimethylamine and ethylene diamine may be used for nucleating compound for sulfuric acid in SAD-RCPC. Stabilizing compounds, such as dimethylamine, react with sulfuric acid at the gas-molecule collision limit and does not appreciably react to form particles with other atmospherically relevant compounds.

### Example 4

The devices and systems according to the present invention may be used to measure vertically-resolved concentrations of nucleation precursor gases in order to understand how and why nucleation occurs above the surface layer at the DOE Atmospheric Radiation Measurement Climate Research Facility (ARM) Southern Great Plains site (SGP).

The devices and systems according to the present invention may mounted on the ARM Tethered Balloon System to measure vertically-resolved sulfuric acid and other nucleation precursor gas at the ARM SGP.

The devices and systems according to the present invention may be used to compare measured precursor concentrations to proximity to sources, atmospheric stability, other meteorological conditions, and aerosol condensation sink to develop a process-level understanding on how these gases were formed, transported, and drive nucleation aloft.

The majority of atmospheric particles that develop into cloud droplets originate from the nucleation of precursor vapors. Aerosol microphysics models have estimated that nucleation produces the majority (>50%) of cloud condensation nuclei at altitudes where low level clouds form (about 1 km) compared the ground level (<10%). Recently, it has been observed significant concentrations of lofted nucleation mode particles (<10 nm in diameter) in diverse locations around the world, suggesting that nucleation is occurring above the surface. Due to the significant impact of nucleation aloft on Earth's radiative budget, Earth system models, such as the DOE-led Energy Exascale Earth System Model (E3SM), plan to include nucleation in order to improve predictions of aerosol properties and their impacts on cloud dynamics. However, nucleation above the atmospheric surface layer (>50 m above ground level, AGL) is poorly understood due to instrument limitations preventing AGL measurements of nucleation precursor gases. Comparison of modeled nucleation precursor concentrations (e.g., sulfuric acid) to sparse ground-level measurements shows a factor of 5-100 times lower modeled concentration. This difference may be expected to be higher given that models do not include most precursors. Furthermore, modeled concentrations may be likely more inaccurate with increasing altitude due to poorly understood turbulent transport, removal via coagulation, and reactions of these compounds above the surface. Therefore, vertical profiles of nucleation precursors may be measured to quantify how nucleation impacts atmospheric aerosol concentrations and Earth's radiative budget.

Without wishing to be bound to any particular theory, nucleation above the surface layer may be predominantly driven by sulfuric acid reacting with a range of stabilizing compounds, similar to the diverse reaction mechanisms observed at ground level. The sulfuric acid nucleation potential model (NPM) may be used to analyze measured vertically-resolved concentrations of sulfuric acid and other precursors and estimate nucleation rates and concentrations of nucleation mode particles at various altitudes. NPM simplifies the numerous sulfuric acid nucleation reactions into a single pathway such that the contributions of stabilizing compounds to nucleation are combined into an effective concentration. As such, the model allows the collective contribution of a complex chemical system for enhancing sulfuric acid nucleation rates to be measured using a condensation particle counter, a transformative application of this instrument to detect specific gases.

The system according to the present invention may be used to detect and analyze nucleation in the boundary layer that impacts cloud properties and thus Earth's radiative budget, as well as land-atmosphere linkages, connecting surface precursor emissions with atmospheric processing. Nucleation precursors also play critical roles in other aerosol processes beyond nucleation as these compounds also impact aerosol growth, aqueous chemistry, and cloud activation. Thus, vertically-resolved measurements of nucleation precursors may broadly improve understanding on how aerosol particles impact Earth's radiative budget.

Atmospheric nucleation is very likely occurring above the surface layer as high concentrations of freshly formed particles have been measured during aircraft and mountain campaigns. It has been observed 10³ cm⁻³ nucleation mode particles (<10 nm in diameter) in the free troposphere above the tropical regions of the Atlantic and Pacific Oceans. It has been reported that elevated concentrations of particles with diameters <50 nm in the lower free troposphere above the Amazon Rainforest. At the Atmospheric Radiation Measurement Southern Great Plains site (ARM SGP), it has been detected a clear increase in concentrations of <16 nm diameter particles from 0 cm⁻³ at 100 m to 10⁴ cm⁻³ at 400 m. Furthermore, several mountain research stations have reported higher frequency of nucleation events, bursts of high concentrations of freshly formed particles, compared to ground-level observations. Combined, these observations strongly suggest that nucleation reactions also occur at higher altitudes and can contribute a significant fraction of total aerosol particle concentrations.

Results from aerosol microphysics models also indicate that upper boundary layer nucleation is a vital source of aerosol particles and produces a majority of cloud condensation nuclei (CCN). For example, the Global Model of Aerosol Processes (GLOMAP) simulations suggest that in the upper boundary layer where low level clouds form, nucleation accounts for 50% of the CCN whereas surface nucleation contributed no more 10%. Recent inclusion of four nucleation reactions schemes into the Weather Research and Forecasting model coupled with Chemistry (WRF-Chem) suggests that nucleation accounts for 50-100% of Aitken mode aerosol number concentration (30-60 nm) at 1 km altitude above the Amazon. Particles formed in the upper boundary layer are particularly important as they have a much higher likelihood of serving as CCN for low clouds compared to those formed in the surface layer or upper troposphere. These clouds have been shown to be the most important for cooling the planet. However, model predictions of vertically resolved aerosol number concentrations, and thus CCN concentrations, are highly uncertain in the boundary layer due to limited understanding on how and which nucleation precursor compounds react as a function of altitude and/or lack of vertically-resolved measurements of nucleation precursors concentrations.

Aerosol models currently estimate vertically resolved nucleation precursor concentrations from emission inventories of specific ground-level sources. Sulfuric acid concentrations are estimated from sulfur dioxide emissions and scaled by its oxidation rates into sulfuric acid. Ground measurements of sulfuric acid from urban, rural, and marine regions indicate that simply scaling sulfur dioxide concentrations is inaccurate as sulfuric acid concentrations also depend on condensation sink, ozone, nitrous oxide, and other potentially unknown factors that vary from region to region. Furthermore, emission inventories are incomplete as they do not include all sources of a nucleation precursor and all potential nucleation compounds such as amines or oxidized organics. It has been estimated that non-inventoried dimethylamine concentrations by scaling ammonia emissions by 0.45-1%, as calculated from previous point-source measurements. However, ground measurements in a variety of locations from the ARM SGP to Beijing, China have demonstrated a highly variable scaling factor between 0-10%. This difference is significant because a factor of 10 increase in dimethylamine concentration would result in 10-10⁴ increase in predicted sulfuric acid nucleation rates. It has also been modeled that amine concentrations from emission inventories and their predicted methyl, dimethyl and trimethyl amine concentrations were 1-2 orders of magnitude below observations. Thus, reducing the uncertainty in nucleation precursor concentrations is required to significantly improve model predictions of nucleation rates and aerosol particle concentrations.

The challenge with measuring nucleation precursors above the ground is that current instruments, specifically the widely used atmospheric pressure, Chemical Ionization Mass Spectrometer (CIMS), are roughly the size and weight of a refrigerator and are too bulky for most aerial measurements. CIMS measures speciated precursor gases and nucleated clusters and have been used to measure sulfuric acid and ammonia during aircraft measurements. However, aircraft measurements only represent one point in time and space and will not capture the dynamics of how nucleation precursors are lofted into the atmosphere and quickly react (often on timescales of minutes to hours). Other potential aerial platforms include unmanned aerial vehicles (UAV) or tethered balloon system (TBS), both of which could remain stationary and provide a clearer picture on how compounds are transported from ground sources to higher altitudes. Unfortunately, mounting a CIMS on an UAV or TBS is not currently feasible due to operational limits on weight and power. Consequently, a new, compact nucleation precursor measurement technique must be developed to observe how nucleation contributes to particle concentrations above the surface layer.

The systems and devices according to the present invention may be used to develop process-level understanding on how and why nucleation occurs above the surface layer by measuring vertically resolved nucleation precursors. Without wishing to be bound to any particular theory, similar to ground level nucleation, sulfuric acid is the key driver for nucleation above the surface layer due to lofting of surface emissions. Sulfur dioxide has been detected at tens of parts per trillion up to 12 km in altitude during the Atmospheric Tomography (ATom) aircraft campaigns and will oxidize into sulfuric acid. Biogenic nucleation also occurs above ground level but likely only in pristine environments, e.g., parts of the Amazon Rainforest and Himalayan mountains. In addition, laboratory measurements and chemical box modeling indicate that biogenic nucleation contributes fewer number of aerosol particles compared to sulfuric acid nucleation in present times. As sulfuric acid and water nucleation alone cannot explain observed particle concentrations, it is believed that other stabilizing compounds such as ammonia and amines must also exist and contribute to nucleation above the surface layer.

A nucleation potential model (NPM) is used to develop a compact instrument to measure sulfuric acid and its stabilizing compounds and quantify sulfuric acid nucleation rates above the surface layer. NPM takes into account the ever-expanding list of stabilizing compounds that react with sulfuric acid to form stable, 1-nm particles. This model groups all stabilizing compounds into one parameter, an effective base concentration ([B_{eff}]), and assumes sulfuric acid reacts in stepwise acid-base reactions to form a 1-nm particle. The consolidated base concentration may be determined from measured concentration of 1-nm particles produced from reacting a known concentration of sulfuric acid with atmospheric air. [B_{eff}] complements the speciated nucleation precursor measurements taken by a CIMS while also capturing the yet unknown intricacies of how a complex chemical system (>4 compounds) nucleates with sulfuric acid. NPM is therefore well-suited to quantify how sulfuric acid nucleates in the atmosphere.

A corollary of NPM is it also allows a 1-nm condensation particle counter (CPC) instead of a CIMS to measure the grouped concentration of stabilizing compounds, as we have Previously done. In addition, the use of NPM may be used to enable measurement of vertically resolved sulfuric acid concentrations with a CPC. A CPC is more robust and easier to calibrate for freshly nucleated particles than a CIMS. CPCs have already been mounted on a wide range of aerial systems because these instruments are lightweight and consume low amounts of power. The systems and devices according to the present invention may be characterized by being a compact nucleation precursor counters capable of being launched on a tethered balloon system (TBS); configured to measure nucleation precursors and particles at various altitudes (0-1000 m) at the ARM SGP using the TBS; and/or detect and analyze measured vertical distribution of nucleation precursors contribution to particle concentrations and the impact on precursor concentration profiles by meteorological conditions and vertical transport of gases.

There is also evidence at the ARM SGP that NPF occurs aloft, followed by transport down to the surface, where particle growth continues to a potentially CCN-active size. To further expand on those earlier observations, an ARM-supported field campaign (Vertically Resolved NPF and Transport) at the SGP during the summer/fall of 2019 and the winter of 2020 measured vertically-resolved concentrations of atmospheric aerosol particles down to 1 nm in order to develop process-level understanding for the formation and growth of atmospheric aerosol aloft. These measurements may related to at least one of the following: because NPF aloft followed by transport of grown particles to the surface may be a significant source of CCN observed at the surface; to connect the atmospheric conditions that drive atmospheric NPF with large-scale boundary layer transport processes and meteorology; and to evaluate the extent to which surface-based aerosol measurements are representative of the atmospheric aerosols aloft since the vast majority of ambient NPF model parameterizations have been based on surface aerosol observations.

The effective concentration of the stabilizing compounds, [B_{eff}], from NPM captures the ability of various compounds to stabilize sulfuric acid clusters, including synergistic effects, and their corresponding concentrations. Compounds that enhance sulfuric acid nucleation rates more (i.e., reduce sulfuric acid evaporation rates per the acid-base model) are represented by higher [B_{eff}] and weaker stabilizing agents reduce [B_{eff}]. Note, [B_{eff}] is not limited to basic compounds as it includes all compounds that could help stabilize sulfuric acid clusters, such as water, organics, and ions. Grouping all potential stabilizing compounds together avoids identifying each compound or combination of compounds in the atmosphere that nucleate with sulfuric acid and quantifying their effects on sulfuric acid nucleation rates. Thus, [B_{eff}] is estimated from known concentrations of sulfuric acid, [A1], freshly produced 1 nm particles, and nucleation reaction time.

The systems and devices according to the present invention may be characterized by a TBS-mountable condensation particle counter that measures concentrations of sulfuric acid and stabilizing compounds; a compact base-CPC and SAD-RCPC deployable on a TBS.

A base-CPC that measures [B_{eff}] is shown in FIG. 5. The base-CPC comprises a sulfuric acid nucleation flow reactor (i.e., constantly purged with nitrogen entrained with gaseous sulfuric acid) with 1 nm, versatile water CPC (vWCPC, TSI 3798). The base-CPC reacts a known amount of sulfuric acid vapor with nucleation precursor.

As shown in FIG. 5, the base-CPC comprises a sulfuric acid flow reactor and a 1-nm vWCPC. Teflon mesh is used to straighten the flow gases in sampled air within a flow reactor to form detectable 1 nm particles. The glass flow reactor is 25 cm long and has an inner diameter of 5 cm. The reactor temperature is held at 300 K and 20% relative humidity (RH). Sulfuric acid vapors are generated by passing clean, humidified nitrogen over a sulfuric acid permeation tube. Sulfuric acid concentration in the flow reactor is measured separately at 5×10⁸ cm⁻³ by an atmospheric pressure, nitrate chemical ionization long time of flight mass spectrometer (CIMS). Sulfuric acid concentration is stable when the reactor is continuously purged with sulfuric acid vapor. In addition, sulfuric acid concentration in the reactor is much higher than atmospheric concentrations which allows the base-CPC to sample outdoor air without significant interference with ambient sulfuric acid. The reaction time between sulfuric acid vapors and injected sampled air is about 2 s, as determined by computational fluid dynamics. A short nucleation reaction time may be desirable to reduce/prevent freshly nucleated clusters from growing beyond 1 nm; and/or minimize non-sulfuric acid nucleation reactions to measured 1 nm particle concentrations. [B_{eff}] is determined from the base-CPC using NPM with inputs of sulfuric acid concentration, measured 1 nm particle concentration, nucleation time, wall loss and coagulation rates.

Referring to FIG. 6, individual and combinations of stabilizing compounds are sampled by the base-CPC, and the resulting [B_{eff}] as a function of measured base concentration. Note, higher [B_{eff}] indicates compound(s) enhance sulfuric acid nucleation more than compound(s) with lower [B_{eff}]. The bases include ammonia, methylamine, dimethylamine, and trimethylamine. True base concentrations are measured by the CIMS using hydronium ion (H3O+·nH2O). FIG. 6 shows combined Base Concentration via CIMS, [B] (pptv), of only the strongest bases (DMA and TMA). The observed [B_{eff}] for this combination is similar to the sum of DMA and TMA concentrations. Results suggest [B_{eff}] is primarily dependent on the concentration of the of the most potent sulfuric acid stabilizing compounds. Thus, the base-CPC may be used to detect pptv (10⁷ molecules cm⁻³) concentrations of DMA and TMA>

### Measurement of Stabilizing Compounds, [Beff]

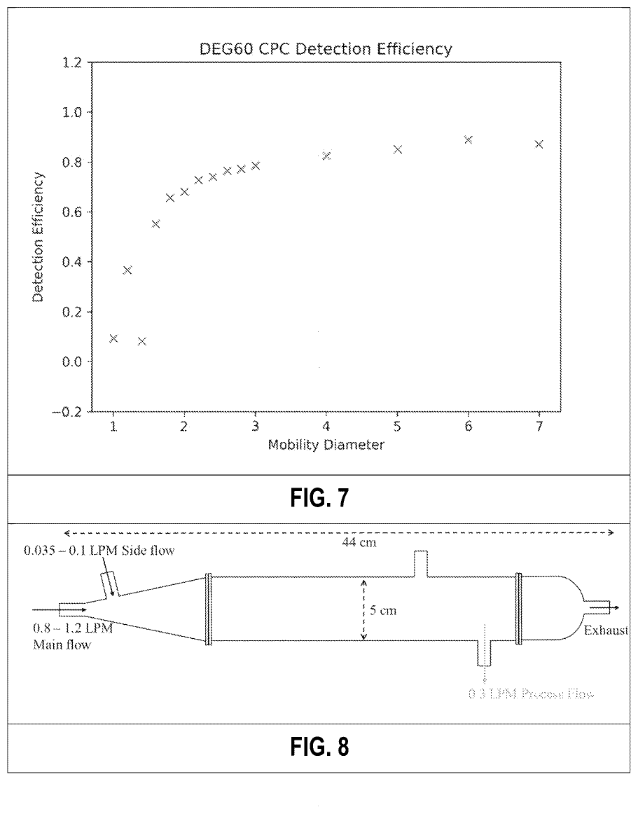

The base-CPC may be configured to measure vertically resolved nucleation precursor concentrations on the TBS at the ARM SGP in view of the instrument weight, power consumption, ruggedness, and performance variability with changes in temperature and relative humidity (RH). The base-CPC may comprise a DEG WCPC which is lighter weight (2.4 kg), smaller (18.5×16.5×21 cm), with lower power consumption, and is operated in any orientation. The DEG WCPC is has particle size detection limit of about 1.6 nm mobility diameter (DEG inlet+WCPC), as shown in FIG. 7.

FIG. 7 shows particle size-dependent counting efficiency of DEG WCPC, with a 50% cut-size lowered to 1.5 nm and 1.9 nm for negatively and positively charged ammonium sulfate (AS), respectively.

The device and system according to the present invention may comprise pulse height analysis (PHA) technique to determine the size and concentration of particles that nucleate in the base-CPC. A large source of uncertainty in measuring [B_{eff}] with the base-CPC is growth of particles outside NPM 1 nm model range. Larger formed particles may increase the coagulation rate loss.

The base-CPC may utilize a source of clean nitrogen which is not possible while deployed on the TBS. As a result, the sulfuric acid flow reactor of the base-CPC may comprise additional pump to pull about 2-4 LPM atmospheric air over the sulfuric acid permeation tube to generate sulfuric acid vapor and sampled air for analysis. Air passing over the sulfuric acid permeation tube is filtered of particles, organics, and other contaminates via activated carbon and molecular sieve filters, and/or compact catalytic strippers with low power requirements. Catalytic strippers produce contaminant-free air; however, their power requirements may reduce base-CPC operation time on a battery to 1-2 hours. The sampled air may be filtered to remove pre-existing particles to reduce the time-resolution in detecting rapid changes in [B_{eff}] as basic gases tend to effectively adsorb then desorb off surfaces over the course of tens of minutes. This delay may be minimized by increasing the sample flow to reduce wall deposition.

The TBS-deployable base-CPC may be used at different temperatures and RH to detect and measure the sulfuric acid permeation rate and ultimately sulfuric acid concentration in the flow reactor changes. CIMS is used to characterize sulfuric acid concentration in the flow reactor as a function of temperature and RH. Also, calculated [B_{eff}] as a function of temperature and RH for a variety of laboratory-generated precursor mixtures is determined as these parameters may influence nucleation rates in the reactor.

The present invention is directed to the following aspects:

Aspect 1. An apparatus to measure gaseous reactive gases that form atmospheric aerosol particles, the apparatus comprising a flow reactor, such as a laminar flow reactor, in fluid communication with a condensation particle counter, such as a water condensation particle counter, wherein the flow reactor comprises a reaction chamber having a first inlet configured to receive a continuous flow of a first fluid and a second inlet configured to receive a continuous flow of a second fluid; a first outlet in fluid communication with the condensation particle counter, and configured to receive a 1-3 nanometer particle comprising a reaction product of the first fluid and second fluid; and a second outlet comprising an exhaust.

Aspect 2. The apparatus of aspect 1, wherein the reaction chamber is cone-shaped.

Aspect 3. The apparatus of any of the foregoing aspects, wherein the first inlet is positioned at a vertex of the reaction chamber.

Aspect 4. The apparatus of any of the foregoing aspects, wherein the vertex of the reaction chamber has an angle from 15-60 degrees, 15-30 degrees, 30-60 degrees, or 35-55 degrees.

Aspect 5. The apparatus of any of the foregoing aspects, wherein the reaction chamber comprises an angle from 15-30 degrees, 30-45 degrees, or 45-90 degrees between a longitudinal axis of the first inlet and a longitudinal axis of the second axis.

Aspect 6. The apparatus of any of the foregoing aspects, wherein the first inlet has a cross-sectional area at least 1-10 times greater than a cross-sectional area of the second inlet, such as, 2 times, 3 times, 4 times, 5 times, 6 times, 7 times, 8 times, 9 times, 10 times, or greater than 10 times, or equal to the cross-sectional area of the second inlet.

Aspect 7. The apparatus of any of the foregoing aspects, wherein the second outlet is coaxial with the first inlet.

Aspect 8. The apparatus of any of the foregoing aspects, wherein the first outlet is perpendicular to the second outlet.

Aspect 9. The apparatus of any of the foregoing aspects, wherein the second inlet comprises a plurality of inlets coaxial with the first inlet, for example, the second inlet may comprise at least two inlets, at least three inlets, at least four inlets, at least five inlets, or at least six inlets.

Aspect 10. The apparatus of any of the foregoing aspects comprising a filter intermediate the first inlet and first outlet to filter/separate particles having a diameter greater than a threshold diameter from particles having a diameter less than or equal to the threshold diameter, wherein the threshold diameter is 3 nanometers, 4 nanometers, 5 nanometers, 6 nanometers, 7 nanometers, 8 nanometers, 9 nanometers, 10 nanometers, or greater than 10 nanometers.

Aspect 11. The apparatus of any of the foregoing aspects comprising a center flow extractor to filter/separate particles other than the reaction product away from the first outlet.

Aspect 12. The apparatus of any of the foregoing aspects, wherein the first fluid comprises one of (a) an atmospheric gas comprising at least one reactant and (b) an amine; and the second fluid comprises the other one of (a) the atmospheric gas comprising at least one reactant and (b) the amine.

Aspect 13. The apparatus of any of the foregoing aspects, wherein the reactant comprises at least one of sulfuric acid, dimethylamine, trimethylamine, and diamine).

Aspect 14. The apparatus of any of the foregoing aspects, wherein the atmospheric gas comprises a carrier gas comprising at least one of water, nitrogen, oxygen, hydrogen, and argon.

Aspect 15. The apparatus of any of the foregoing aspects, wherein the amine comprises at least one of dimethylamine, ethylenediamine, trimethylamine, and 1,4-butanediamine.

Aspect 16. The apparatus of any of the foregoing aspects, wherein the reaction product comprises at least one of sulfuric acid-dimethylamine particles, sulfuric acid-ethylene diamine particles, sulfuric acid-trimethylamine particles, and sulfuric acid-1,4-butanediamine particles.

Aspect 17. The apparatus of any of the foregoing aspects, wherein the reactant has a concentration from 1×10⁶ to 8×10⁸ molecules cm⁻³.

Aspect 18. The apparatus of any of the foregoing aspects, wherein reaction chamber has a temperature from 21-25° C.

Aspect 19. The apparatus of any of the foregoing aspects, wherein the reaction chamber has a relative humidity from 5-90%.

Aspect 20. The apparatus of any of the foregoing aspects, wherein the reaction product has a rate of 1-30 seconds, 10-24 seconds, 1-3 seconds.

Aspect 21. The apparatus of any of the foregoing aspects, wherein a distance from the second inlet to the first outlet is sufficient to generate a residence time of the reaction product (i.e., a time sufficient for the first fluid to react with the second fluid to generate the reaction product) in the reaction chamber from 1-30 seconds, 10-24 seconds, or 1-3 seconds.

Aspect 22. The apparatus of any of the foregoing aspects wherein the first fluid has a flow rate from 35 cm³/min to 1 L/min and the second fluid has a flow rate from 35 cm³/min to 1 L/min.

Aspect 23. The apparatus of any of the foregoing aspects comprising a diethylene glycol saturator in fluid communication with and coupled to the condensation particle counter.

Aspect 24. The apparatus of any of the foregoing aspects comprising a dual channel diethylene glycol water condensation particle counter comprising a first channel to measure ambient (e.g., outdoor) particle concentration and a second channel to measure ambient (e.g., outdoor) particle concentration and the reaction product.

Aspect 25. The apparatus of any of the foregoing aspects comprising a third outlet in fluid communication with the first channel and the first outlet is in fluid communication with the second channel.

Aspect 26. The apparatus of any of the foregoing aspects tethered to a balloon.

Aspect 27. The apparatus of any of the foregoing aspects coupled to an aerial drone.

Aspect 28. A method of measuring atmospheric reactive precursor gases that form aerosol particles, the method comprising: contacting a continuous flow of a first fluid and a continuous flow of a second fluid in a reaction chamber of a flow reactor to generate a 1-10, 1-2, 1-3, or 2-3 nanometer particle comprising a reaction product of the first fluid and second fluid; and detecting the particle via a condensation particle counter.

Aspect 29. The method of any of the foregoing aspects comprising separating the reaction product from particles other than the reaction product.

Aspect 30. The method of any of the foregoing aspects, wherein the nanometer particle consists essentially of the reaction product of the first fluid and second fluid.

Aspect 31. The method of any of the foregoing aspects, wherein the nanometer particle consists of the reaction product of the first fluid and second fluid.

Aspect 32. The method of any of the foregoing aspects, wherein the first gas comprises atmospheric sulfuric acid gas, the second gas comprises dimethylamine or ethylene diamine gas, and the reaction product comprises a sulfuric acid-dimethylamine particle or sulfuric acid-ethylene diamine particle.

Aspect 33. The method of any of the foregoing aspects, wherein the first gas comprises atmospheric sulfuric acid gas, the second gas comprises dimethylamine or ethylene diamine gas, and the reaction product consists essentially of a sulfuric acid-dimethylamine particle or sulfuric acid-ethylene diamine particle.

Aspect 34. The method of any of the foregoing aspects, wherein the first gas comprises atmospheric sulfuric acid gas, the second gas comprises dimethylamine or ethylene diamine gas, and the reaction product consists of a sulfuric acid-dimethylamine particle or sulfuric acid-ethylene diamine particle.

Aspect 35. The method of any of the foregoing aspects wherein the first gas comprises atmospheric dimethylamine, trimethylamine, or other strong bases like diamines, the second gas comprises of sulfuric acid, and the reaction product comprises strong basic compound-sulfuric acid particles.

Aspect 36. The method of any of the foregoing aspects wherein the first gas comprises atmospheric dimethylamine, trimethylamine, or other strong bases like diamines, the second gas comprises of sulfuric acid, and the reaction product consists essentially of strong basic compound-sulfuric acid particles.

Aspect 37. The method of any of the foregoing aspects wherein the first gas comprises atmospheric dimethylamine, trimethylamine, or other strong bases like diamines, the second gas comprises of sulfuric acid, and the reaction product consists of strong basic compound-sulfuric acid particles.

Aspect 38. A method of measuring atmospheric reactive precursor gases that form aerosol particles using the apparatus of any of the foregoing aspects.

Aspect 39. A method of measuring atmospheric reactive precursor gases that form aerosol particles comprising contacting a continuous flow of atmospheric sulfuric acid and a continuous flow of dimethylamine for a residence time to generate a reaction product comprising 1-2 nanometer sulfuric acid-dimethylamine particles; and detecting the 1-2 nanometer sulfuric acid-dimethylamine particles via a condensation particle counter.

Aspect 40. The method according to any of the foregoing aspects, wherein the first fluid comprises an atmospheric gas comprising at least one reactant; wherein the second fluid comprises a stabilizer that reacts with and nucleates the at least one reactant; and wherein the stabilizer does not substantially react with and nucleate atmospheric reactive precursor gases other than the at least one reactant during a residence time sufficient of generate the reaction product from 10-24 seconds. In other words, the reaction product is substantially free, essentially free, or completely free from reaction products other than 1-2 nanometer sulfuric acid-dimethylamine particles because the stabilizer does not substantially react with and nucleate atmospheric reactive precursor gases other than the at least one reactant during the residence time.

Aspect 40. The method according to any of the foregoing aspects comprising separating the 1-2 nanometer sulfuric acid-dimethylamine particles from atmospheric particles having a diameter greater than 7 nanometers prior to detecting the 1-2 nanometer sulfuric acid-dimethylamine particles.

All documents cited herein are incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications, and substitutions. This application including the appended claims is therefore intended to cover all such changes and modifications that are within the scope of this application.

What is claimed is:

1. A method of measuring atmospheric reactive precursor gases that form aerosol particles, the method comprising:
contacting a continuous flow of a first fluid and a continuous flow of a second fluid in a reaction chamber of a flow reactor to generate a 1-2 nanometer particle comprising a reaction product of the first fluid and second fluid; and
detecting the particle via a condensation particle counter.

2. The method of claim 1, wherein the first fluid comprises an atmospheric gas comprising at least one reactant.

3. The method of claim 2, wherein the reactant comprises at least one of sulfuric acid, dimethylamine, trimethylamine, and diamine.

4. The method of claim 1, wherein the reactant has a concentration from 1×10⁶ to 8×10⁸ molecules cm⁻³.

5. The method of claim 1, wherein the first fluid comprises a carrier gas comprising at least one of water, nitrogen, oxygen, hydrogen, and argon.

6. The method of claim 1, wherein the second fluid comprises at least one of dimethylamine, ethylenediamine, trimethylamine, and 1,4-butanediamine.

7. The method of claim 1, wherein the reaction product comprises at least one of sulfuric acid-dimethylamine particles, sulfuric acid-ethylene diamine particles, sulfuric acid-trimethylamine particles, and sulfuric acid-1,4-butanediamine particles.

8. The method of claim 1 comprising generating laminar flow of at least one of the first fluid, second fluid, and reaction product.

9. The method of claim 1 comprising turbulent flow of at least one of the first fluid, second fluid, and reaction product.

10. The method of claim 1 comprising mixing the first fluid and second fluid for 10-24 seconds to generate the reaction product.

11. The method of claim 1 comprising separating the reaction product from particles other than the reaction product.

12. The method of claim 1 comprising separating particles having a diameter greater than or equal to 7 nanometers from particles having a diameter less than 7 nanometers.

13. The method of claim 1, wherein the particle detected via the condensation particle counter comprises a diameter from 1-2 nanometers.

14. The method of claim 1, wherein reaction chamber has a temperature from 21-25° C.

15. The method of claim 1, wherein the reaction chamber has a relative humidity from 5-90%.

16. The method of claim 1, wherein the first fluid has a flow rate from 35 cm³/min to 1 L/min and the second fluid has a flow rate from 35 cm³/min to 1 L/min.

17. The method of claim 1, wherein the reaction chamber is tethered to one of a balloon and an aerial drone.

18. A method of measuring atmospheric reactive precursor gases that form aerosol particles, the method comprising:
contacting a continuous flow of atmospheric sulfuric acid and a continuous flow of dimethylamine for a residence time from 10-24 seconds to generate a reaction product comprising 1-2 nanometer sulfuric acid-dimethylamine particles; and
detecting the 1-2 nanometer sulfuric acid-dimethylamine particles via a condensation particle counter.

19. The method of claim 18,
wherein the first fluid comprises an atmospheric gas comprising at least one reactant,
wherein the second fluid comprises a stabilizer that reacts with and nucleates the at least one reactant to generate the reaction product; and
wherein the stabilizer does not substantially react with and nucleate atmospheric reactive precursor gases other than the at least one reactant during the residence time.

20. The method of claim 18 comprising separating the 1-2 nanometer sulfuric acid-dimethylamine particles from atmospheric particles having a diameter greater than 7 nanometers prior to detecting the 1-2 nanometer sulfuric acid-dimethylamine particles.
